(19) United States
(12) Patent Application Publication
(10) Pub. No.: US 2022/0257404 A1
(43) Pub. Date: Aug. 18, 2022

(54) GARMENT FOR PROMOTING POST-SURGICAL RECOVERY

(71) Applicants: Thomas E. BROWN, Ripon, CA (US); Diane K. BROWN, Ripon, CA (US)

(72) Inventors: Thomas E. BROWN, Ripon, CA (US); Diane K. BROWN, Ripon, CA (US)

(21) Appl. No.: 17/736,021

(22) Filed: May 3, 2022

Related U.S. Application Data
(63) Continuation of application No. PCT/US2020/059125, filed on Nov. 5, 2020.
(60) Provisional application No. 62/931,730, filed on Nov. 6, 2019.

(51) Int. Cl. A61F 5/03; A61F 5/32

(52) U.S. Cl. CPC A61F 5/03; A61F 5/32

(57) ABSTRACT

A compression fabric garment with a low vertical profile, composed from comfortable and breathable compressive fabric, with high-strength polymer quick-release tension bands, and antimicrobial fabric portions is disclosed. Embodiments of the present disclosure may include a post-surgical compression garment for relief after open-heart surgery, mastectomy, abdominal surgery, or other surgery, equipped with at least one quick-release tension band to apply tension laterally across the body where the front of the garment is configured to flip downwards to provide quick access to a chest, or other, surgical incision site without fully removing the garment.

### CROSS-REFERENCE TO RELATED APPLICATIONS

The present application claims priority to and benefit of PCT Patent Application Serial No. PCI/US2020/059125 filed on Nov. 5, 2020, which claims priority to and benefit of provisional U.S. application No. 62931730 filed on Nov. 6, 2019.

### TECHNICAL FIELD

The invention relates to garments and braces adapted for pain relief and promoting patient recovery following surgery, and in particular a compression garment adapted for quick and easy access to the patient's chest and a surgical site, facilitating treatment and reducing infection of wounds of a patient after having open-heart surgery, Cesarean section surgery, abdominal surgery, mastectomy, or other surgery on the torso.

### BACKGROUND ART

Following open-heart surgery, when a patient's breastbone has been cut through to expose the heart, patients have experienced long and painful recoveries as the heart, severed bone, and surgical incisions have healed. Electrical leads which run through the chest and connect directly to the heart to facilitate revival of a patient with the use of a defibrillator remain exposed and protrude out of the chest. Following surgery, tubing is also inserted through the abdomen and into the lungs, which remains exposed, to facilitate fluid drainage outside the body, and to ensure proper breathing.

As patients cough, sneeze, and complete other basic bodily movements following surgery, pressure placed on the chest at both sides of the incision causes movement between the severed portion of the breastbone and the adjacent bone, which is both painful and harmful to patient recovery. Devices such as the Heart Hugger™, described in U.S. Pat. No. 4,641,642, have generally provided for loosely positioned immobilizing harnesses. Such devices have offered a more limited ability to facilitate several aspects of improved patient recovery and have not been designed to use immediately post-surgery while the patient has still been in the hospital. Thus, it will be appreciated that prior art thorax support devices have not provided adequate quick access to the surgical site, access to leads coming through the chest wall for emergency resuscitation, or adaptation for drainage tubes exiting from the chest wall. Such prior art thorax support devices have been unsuitable for use in the days immediately following surgery while a patient has still been in the hospital with tubes and leads extending from the patient's chest wall, and such prior art support devices also have been otherwise unsuitable for longer term patient recovery.

As noted by Applicant, such prior art thorax support devices, such as the Heart Hugger, have been hard to operate and have not provided sufficient compressive force to the patient's thorax in a way so as to adequately reduce pain at a surgical site and to promote recovery. Such prior art thorax support devices have also been difficult to remove, and they have not provided quick access to the chest and surgical sites in the event of an emergency. Other prior art devices have been too large and bulky to be comfortably worn immediately following surgery while a patient is bedridden. Such large and bulky devices have also been inadequate for longer term recovery because of the difficulty in positioning them over or under clothing, and because of discomfort they have caused when patients have been required to fit into tight spaces such as a car.

Other types of surgery on the torso, such as abdominal surgery or Cesarean section surgery, also cause pain as patients cough, sneeze, and complete other basic bodily movements following surgery. Abdominal binding devices such as U.S. Pat. No. 7,160,262 B2, for Abdominal Binding, to Wicks, and U.S. Pat. No. D752,233S, for Postpartum Abdominal Support, to Fruscione-Loizides, have been used to encircle the abdomen and provide compression for pain relief, stability of the surgical site, and back support. However, these vests and abdominal binders have not necessarily been specifically designed for surgical wound support in the thorax or abdomen, have been uncomfortably tight at times, and have caused discomfort around the surgical site and skin irritation. Further, such devices may provide too much compression or too little, whereas other such prior art devices have blocked heat and air exchange between the enwrapped skin and the atmosphere, raising the body temperature around the encased area and causing perspiration around the wound site. Therefore, there is a need for improvement and more breathable and comfortable support around abdominal surgical sites to enable better recovery.

After a mastectomy, sometimes a chest area is bound immediately post-surgery with breast binders, and patients are thereafter encouraged to wear a post operation bra or continue to use some form of breast binders to stabilize the wound, provide support, provide pain relief, and facilitate wound healing. Mastectomy garments such as U.S. Pat. No. 10,681,942 B2, Reconfigurable Post-Surgery Brassiere, to Thompson, and U.S. Pat. No. 10,357,065 B2, Post Surgery Brassiere Garment, to Donlon et al., have provided improved and more breathable binders. However, these prior art devices have been designed as brassieres and do not cover the whole body to provide equalized tension around the entire torso of the body, including upper and lower portions of the chest, the shoulder blades and the back, while giving quick access to the wound site. Further, these prior devices have not allowed a wearer to choose to whether to apply the compression on an upper portion of the breasts, a lower portion of the breasts, on both regions, on either side of the chest, or over the entire chest, as a wearer my consider desirable.

Prevention of infection in surgical sites following surgery has also been a major focus of patient recovery. Following surgery, patients are ordinarily treated with antibiotics and instructed to keep a wound clean in order to prevent infection. However, even with antibiotic therapy and proper wound care, it is still possible for a surgical site to become infected. As disclosed in U.S. Pat. No. 7,329,417, to Nielsen, for Medical Dressing Comprising an Antimicrobial Silver Compound, silver compounds have been known for dressing wounds, and as disclosed in US Patent Application No. 2016/0361190, to Caden et al., for Undergarment, incorporating antimicrobial silver compounds into a garment to prevent infection has been developed in the prior art. However, prior art antimicrobial silver compounds have not been incorporated into garments and braces adapted for pain relief and promoting patient recovery for use immediately following open-heart or other types of surgeries. Thus, where multiple surgical incisions have been made, as for example in a double mastectomy surgery, there has not been provided a sufficiently-tailored system of addressing such multiple varied surgical incisions as part of a pain reducing post-surgery support or compression garment. Therefore, a better garment for post-surgery recovery is needed to address some of the above-mentioned problems.

### DISCLOSURE OF INVENTION

In response to the foregoing described limitations of prior art devices relating to post-surgical access to surgical sites, and limitations relating to reducing pain and facilitating improved patient recovery, and in accordance with one or more aspects of the present disclosure, a comfortable garment with a low vertical profile, which provides a substantial and adjustable compressive force to the area surrounding the wearer's surgical site, and which is adapted for quick exposure of the patient's chest for emergency resuscitation by doctors or other hospital staff, for easier treatment of the surgical site and the like, and which prevents infection of surgical incisions located right at where the garment is to be opened and attachable, is disclosed.

Embodiments of the present disclosure may comprise a post-surgical compression, that is also supporting, garment comprising a garment body with a front and rear garment portions which wrap around the torso of a wearer, covering the chest to and surgical incision site. The garment body has arm holes. The garment forms a first and a second front edges, with the garment configured to open down the middle with a fastener, further equipped with at least one quick-release tension band to apply tension laterally across the body, and with at least one quick-release upper strap at the shoulder region applying tension from the front of the garment to the rear of the garment. The quick-release tension band and the quick-release upper strap, in tandem with the positioning of the arm holes, beginning at a level near the bottom of the user's sternum, allow the front of the garment to flip downwardly to provide quick access to the chest and surgical incision sites around the upper torso area without fully removing the garment. In an embodiment, the at least one quick-release upper strap may preferably be two quick-release upper straps, each positioned adjacent a shoulder region of the patient during use. The quick-release upper straps may be elastic and provide tension along a vertical axis between the back of the garment body and the front of the garment body. The quick-release mechanism of the quick-release upper straps may be hook-and-loop fasteners. The hook-and-loop fasteners may be lengthened as to allow for control over the amount of tension provided through the positioning of the quick-release upper straps. Other means of attaching the quick-release upper straps that are known in the art may also be used. Lowering of the front garment portion may be facilitated by releasing of the quick-release tension upper strap(s) as well as the quick-release tension band.

In embodiments of the present disclosure, at least one quick-release tension band may be two quick-release tension bands positioned laterally of the body. The first quick-release tension band may attach adjacent to the first front edge and a second quick-release tension band may attach adjacent to the second front edge to provide equalized tension for the wearer. The attachment mechanism for the quick-release tension bands may be a hook-and-loop, or loop-and-hoop, fastener or any other similar means of attachment that allows the wearer to control the amount of compression the quick-release tension bands provide. Further, a first chest handle may be placed vertically over the first quick-release tension band and a second chest handle 5 may be placed vertically over the second quick-release tension band adjacent respective front edges of the garment body. The chest handles may allow a wearer to apply self-imitated compression by pulling the chest handles close together. Chest handles may also be utilized by a caregiver to adjust, move, or stabilize the patient. In an embodiment, a first side handle and a second side handle may be attached, each below an arm hole. These side handles may allow a caregiver to stabilize or adjust the patient from the side and may assist in preventing falls.

In embodiments of the present disclosure, the rear garment portion may comprise first and second shoulder tension bands that each begin and extend from one of the two quick-release upper straps to form a cross over the shoulder blades of the wearer to create support and equalize tension across the wearer's back, and to help with a better posture. Each shoulder tension band may preferably end under the arm holes that are placed at the opposing side of the quick-release upper straps each shoulder tension band began from.

In embodiments of the present disclosure, the rear garment portion may comprise a first back tension band that is positioned laterally across the rear garment portion to lay across the back of the wearer to provide support in that region, and a second back tension band that is positioned laterally across the mid-back area of the rear garment portion so as to lay across the mid-back of the wearer to provide support in that region.

In embodiments of the present disclosure, the garment body may comprise multiple (e.g., dual) layers so as to provide more compression, and it may be adapted to wrap at least around the thorax of the wearer with the front garment portion positioned at least over a surgical incision site near the sternum of the wearer. The quick-release tension band can be two quick-release tension bands placed around the chest that ends adjacent to each of the front edges. The quick-release tension band and the quick-release upper straps, in tandem with the positioning of the arm holes, beginning at a level near the bottom of the user's sternum, allow the front of the garment to flip downwardly to provide quick access to the chest and surgical incision sites around the chest area without fully removing the garment.

In embodiments of the present disclosure, the fastener that holds the two front edges together may be comprised of at least one zipper. In an embodiment, at least one zipper may preferably be dual-separating zippers so a surgical incision site around lower torso can be quickly accessed by pulling upward a zipper without unzipping the top portion of the garment or fully removing the garment. The zipper material preferably may be plastic, so to allow medical imaging of patients without the removal of the garment. In an embodiment, at least one strip of hook-and-loop fastener may attach on the front garment portion adjacent a zipper end to keep the flap of a zipper closed at the bottom of the garment body. An inside zipper liner attached to one of the front edges may wrap around the head of a zipper to decrease friction between the zipper and a wearer's skin.

This embodiment of the present disclosure may also alternatively be configured with an opening centrally positioned at the front of the garment to facilitate the passage of drainage tubes and other leads, such as leads connected directly to the heart through the chest wall, for the patient in the days immediately following surgery. The centrally positioned opening may be covered with an attachable cover once the drainage tubes and other leads are no longer needed. The cover can attach by hook-and-loop fasteners or any other similar means of securing the cover over the opening. The opening cover may also have antimicrobial pads, described below, attached to prevent infection of the incision site where the drainage tubes where taken out of. The tension straps and bands may be produced from a polymer material which is partially non-elastic and partially elastic in another area of the strap/band and has a high tensile strength. The quick-release fasteners attached to the straps/bands may be high-strength hook-and-loop fasteners. The fastener at the front of the garment preferably comprises one or more zippers.

Alternative embodiments of the present disclosure may also be adapted for facilitating recovery of patients in the days and weeks following surgery where they may no longer have tubes exiting through the patient's chest wall or abdomen, but where the patient may still require a compressive support surrounding the wound site wherein primarily the sternum bone was damaged during the surgery. In such embodiments, the garment may be machine washable and produced from a blend of highly compressive, yet lower profile, fabrics which are comfortable and provide adequate ventilation.

Another alternative embodiment of the present disclosure may have, in place of at least one quick-release tension band, multiple quick-release tension bands sewn together into two panels that each attach below respective arm holes of the garment body, wherein the two panels wrap around the abdomen of the wearer and attach together over the two front edges of the garment body. The two panels may be attached together by a hook-and-loop fastener or any other means known in the art.

Embodiments of the present disclosure may further include replaceable antimicrobial pads positioned on the interior of the garment, preferably in an embodiment at the location where the garment is to be openable and closeable with the fastener, but also at other locations conforming to unique patterns of incisions depending upon the type of surgery involved, and which therefore serve to promote healing by keeping the surgical incisions covered and to prevent infection even at a location that is immediately adjacent an openable and closeable area of the garment to facilitate access to the surgical site. Thus, there may be incorporated a liner, such as a vest-shaped liner garment, such as may be made of a stretchable silk/polyester fabric, or mesh, serving as a structural support for locating infused antimicrobial elements therein, or for applying antimicrobial pads at precise locations needed and corresponding to specific incision patterns associated with a particular type of surgery. Thus, for example, an inner lining silk/polyester, or mesh, vest is provided, capable of being sewn or otherwise attached to inner portions of any of the post-surgery support garment embodiments hereof, to which antimicrobial infused areas, antimicrobial sections of fabric, or antimicrobial removable pads, may be incorporated. The antimicrobial agent utilized may be a silver-based antimicrobial. And incorporation of antimicrobial infused areas may provide a softer fabric surface for patients who, for example, have extra sensitive skin resulting from radiation therapy.

Embodiments of the present disclosure may also be elastic and adjustable such that they will be suitable for wearers of varying body sizes, while still preserving the tension and quick access capabilities of the garment.

Embodiments of the present disclosure may also include one or more telemetry pockets for storing electronic monitoring devices attached to the wearer, with overlapping passages in the garment for wires to pass through.

In embodiments of the present disclosure, the post-surgical compression garment may have bra portions, or pads, adapted for female anatomy. The front garment portion may also comprise breast cutouts that are adapted to allow exposure of breasts and are covered by breast covers that are attached to the front garment portion. Breast covers may flip downwards to reveal the breasts in the breast cutout to allow for breast feeding without removal of the garment. Breast covers may be partially sewn into the front garment, or they may be completely detachable. The attachment mechanism may be hook-and-loop fastener or any other means used and known in the art. Additional embodiments of the present disclosure may also include other designs or 5 components necessary to adapt the garment for varying types of anatomy and/or female anatomy.

Embodiments of the present disclosure may have four quick-release tension bands positioned laterally of the body. The first quick-release tension bands may attach adjacent to the first front edge and the second quick-release tension band may to attach adjacent to the second front edge to provide equalized tension for the wearer. The first and the second quick-release tension bands may be positioned laterally to the upper portion of the breasts of the body to give compression to the upper portion of the breasts. The third quick-release tension band may attach adjacent to the first front edge and the fourth quick-release tension band may attach adjacent to the second front edge to provide equalized tension for the wearer. The third and the fourth quick-release tension bands may be positioned laterally to the lower portion of the breasts of the body to give compression to the lower portion of the breasts. The attachment mechanism may be by hook-and-loop, or loop-and-hoop, fastener or any other similar means to allow the wearer to control the amount of compression they desire. A soft, power mesh fabric may be added to line the upper portion of the inside of the front garment portion for additional breathability and comfort for the wearer. Further, multiple layers (e.g., two) of garment fabric may be added as desired for a more firm compression of the garment. Twill tapes may be sewn or attached to the inner sides of the garment body to provide further support.

The various aforementioned embodiments and aspects of the post-surgical support garments of the invention further address the problems with the prior art since they better promote healing with a comfortable garment having a low vertical profile, which provides sufficient and appropriate compressive force to help retain an incision in place as sutured in the wearer's thorax and torso regions, which facilitates quick exposure to (and application of antimicrobial treatments to) the patient's chest and other surgical sites by emergency personnel in the hours and days immediately following surgery and during recovery, which particularly, regarding open-heart surgery, abdominal surgery, Cesarean section surgery and mastectomy, gives such quick and easy access, and which prevents infection of surgical incisions, is disclosed.

The foregoing aspects and embodiments of the disclosure address the weaknesses set forth regarding the prior art pertaining to a compressive garment that not only provides support for relief of pain and healing, but which also enables quick 5 access to the surgical site and chest to facilitate emergency treatment during critical days of post-operative care, while preventing infection.

### BRIEF DESCRIPTIONS OF DRAWINGS

FIG. 1 shows a front view of an embodiment of a garment in accordance with one or more aspects of the disclosure suitable for use immediately following surgery while a patient is still in the hospital and having tubing and wire leads extending to the exterior of the thoracic wall.

FIG. 2 shows an inside-out, front view of the embodiment shown in FIG. 1.

FIG. 3 shows a front view of an embodiment of a garment in accordance with one or more aspects of the disclosure suitable for home and work use in the weeks following surgery once a patient is partially recovered and no longer has tubes or leads extending through the thoracic wall and with one quick-tension release band attached laterally to the body in a front portion of the garment.

FIG. 4 shows an inside-out, front view of the embodiment shown in FIG. 3, adapted with a covering for the opening previously made available for tubes to pass through and suitable for home and work use in the weeks following surgery.

FIG. 5 shows a rear view of an embodiment of a garment in accordance with one or more aspects of the disclosure which utilizes a pair of back tension bands to compress the back and mid-back regions upon fastening of a front opening of the garment.

FIG. 6 shows a close-up front view of a garment in accordance with one or more aspects of the disclosure wherein a tension band which provides a compressive force across the thorax is engaged.

FIG. 7 shows a close-up, inside-out, front view of the embodiment shown in FIG. 1, demonstrating attachment of an antimicrobial pad.

FIG. 8 shows a close-up view of an adjustable, quick-release, hook-and-loop upper strap of one or more embodiments of the disclosure.

FIG. 9 shows a close-up view of an alternative adjustable, quick-release upper strap of present embodiments of the disclosure.

FIG. 10 shows an inside-out, front view of an embodiment of a garment in accordance with one or more aspects of the disclosure, with bra pads adapted for a female anatomy.

FIG. 11 shows a close-up view of the telemetry pocket opening of an embodiment of the disclosure.

FIG. 12 shows a garment in accordance with an embodiment of the disclosure and being worn by a person immediately following surgery while still having tubes passing through the abdomen or chest wall and electrical leads attached directly to the heart.

FIG. 13 shows a garment in accordance with one or more aspects of the disclosure being worn by a person sometime following surgery and in its flipped down state, having provided quick access to the chest and surgical site for medical personnel.

FIG. 14 shows a front view of a garment in accordance with one or more aspects of the disclosure with two quick-release tension bands on the front portion of the garment body, attached to the front portion by a hook-and-loop fasteners. The embodiment further includes chest handles, side handles, dual-separating zippers, and a telemetry pocket.

FIG. 15 shows a front view of a garment in accordance with one or more aspects of the disclosure, showing two quick-release tension band panels that wrap around the abdomen of the wearer and having a dual-separating zipper, to be worn after an abdominal surgery.

FIG. 16 shows a front view of the garment in FIG. 15, with the two quick-release tension panels laid open to the side of the garment body to demonstrate the underside of the panels.

FIG. 17 shows a rear view of a garment in accordance with one or more aspects of the disclosure, demonstrating a pair of back tension bands to compress the back and mid-back regions upon fastening a front opening of the garment, a pair of side handles, and a pair of shoulder tension bands crossing at the middle of the shoulder blade region of the wearer to compress the region upon fastening a front opening of the garment.

FIG. 18 shows a front view of a garment in accordance with one or more aspects of the disclosure, demonstrating two quick-release tension band panels that wrap around the abdomen of the wearer, extended loop-and-hook upper fastener surface for comfort and adjustment of the quick-release upper straps, a strip of hook-and-loop fastener to keep the zipper flap closed, and two breast covers that cover the breast cutouts in the front garment portion, to be worn by a nursing mother after a Cesarean section surgery.

FIG. 19 shows a front view of FIG. the embodiment of FIG. 18 with the breast covers flipped downwards to reveal the breast cutouts.

FIG. 20 shows an inside-out front view of a garment in accordance with one or more aspects of the disclosure, to be worn by a nursing mother after a Cesarean section surgery. FIG. 20 has different quick-release fasteners for the upper quick-release straps than FIGS. 18 and 19, and demonstrates breast cutouts, the zipper liner, and the antimicrobial pad.

FIG. 21 shows an inside-out front view of a garment in accordance with one or more aspects of the disclosure, to be worn by a mother after a Cesarean section surgery. Instead of breast cutouts, bra pads are present along with the antimicrobial pad and the zipper liner.

FIG. 22 shows a front view of the embodiment in FIG. 21 with the quick-release tension band panels open to the side of the garment body to demonstrate the underside of the quick-release tension band panels and the strip of hook-and-loop fastener that prevents the zipper flip from inadvertently opening.

FIG. 23 shows a patient wearing the embodiment of FIG. 14 and engaging in self-imitated compression using the two chest handles.

FIG. 24 shows a patient wearing the embodiment of FIG. 15 and FIG. 16 with the dual-separating zipper pulled upwards to unzip and quickly reveal the antimicrobial pad and the surgical site on the abdomen of the patient without removal of the garment.

FIG. 25 shows a patient wearing the embodiment of FIG. 18 and FIG. 19.

FIG. 26 shows a front view of a garment in accordance with one or more aspects of the disclosure, to be worn after a mastectomy. A pair of quick-release tension bands wrap around the upper portion of the breasts, each band over one breast, and another pair of quick-release tension bands wrap around the lower portion of the breasts, each band over one breast.

FIG. 27 shows a front view of the embodiment of FIG. 26, with one side of the front portion of the garment open to illustrate the inside of the front portion of the garment to demonstrate the zipper liner and power mesh fabric over the upper region of the inside of the front portion of the garment.

FIG. 28 shows a front perspective view of an inner silk/polyester, or mesh, lining for a post-surgery support garment and having a pattern of antimicrobial infused areas (e.g., antimicrobial infused fabric or pads) attached thereto corresponding to incisions for a double mastectomy.

### MODES FOR CARRYING OUT THE INVENTION

As it is used in this disclosure, the word intubated refers to a patient who has been fitted with tubes extending outward from the abdomen primarily to facilitate drainage of fluid away from the body and to otherwise ensure safe breathing, drainage, and healing of the patient.

The various aforementioned embodiments and aspects of the post-surgical support garments of the invention provide a comfortable garment having a low vertical profile, which provides sufficient and appropriate compressive force to help retain an incision in place as sutured in the wearer's thorax and torso regions, which facilitates quick exposure to the patient's chest and other surgical sites on the torso by emergency personnel in the hours and days immediately following surgery and during recovery, which particularly, regarding open-heart surgery, abdominal surgery, Cesarean section surgery and mastectomy, gives such quick and easy access to hospital staff and emergency personnel, and which prevents infection of surgical incisions, is disclosed.

Thus, as shown in the Figures, and in accordance with the teachings of this disclosure, a thin fabric post-surgical compression garment having a low vertical profile, composed of comfortable and breathable compressive fabric, wherein there are also provided high-strength polymer quick-release tension bands, is provided.

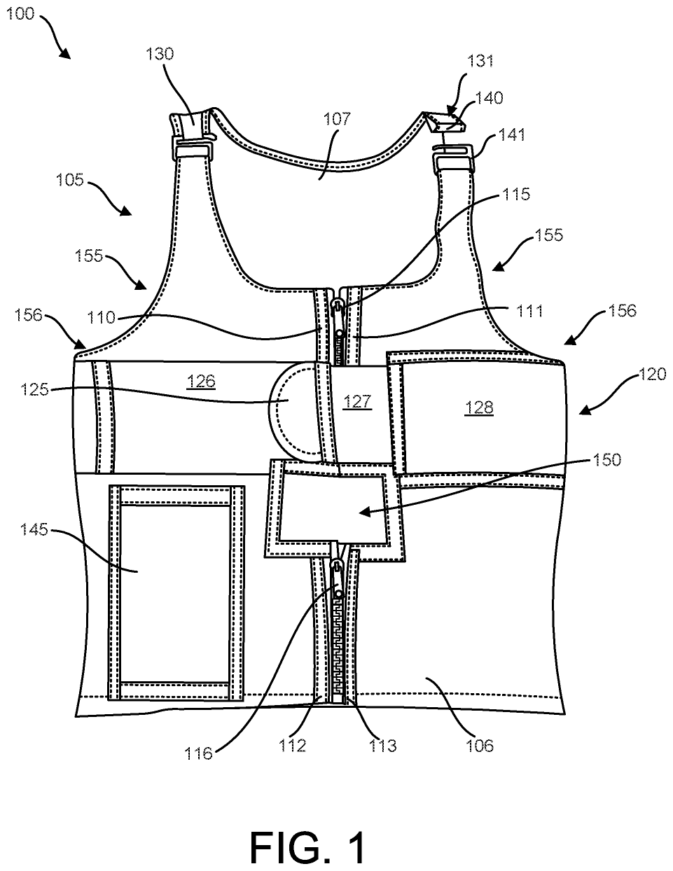

FIG. 1 shows a post-surgical compression garment 100 suitable for use immediately following surgery while a patient is still in the hospital and intubated. The post-surgical compression garment 100 may be fashioned in the shape of a vest with a front portion 106 and a rear portion 107 comprising the garment body 105. The garment body 105 may be configured to open down the middle forming a first front edge no and a second front edge 111, connected by a fastener 15. The fastener 115 may be a zipper 115, or series of zippers 115, 116 as shown. The garment body 105 may define arm holes 155 having a bottom portion 156 of which are preferably adapted to be positioned to start approximately laterally from the bottom of the wearer's sternum bone. In this post-surgical embodiment of the garment 100, the garment body 105 may have a centrally positioned opening 150 at the front portion 106 of the post-surgical compression garment 100 to allow for the passage of breathing tubes leading to the lungs for patients intubated in the hours and days following surgery and such that there are two sets of front edges 110, 111, 112, 113 with fasteners 115, 116, one set of front edges 110, 111 above the opening 150, and one set of front edges 112, 113 below it as shown. Attached to the garment body 105 is at least one quick-release upper strap 130 adapted to be positioned adjacent a patient's should during wear, which applies tension from the back portion 107 of the garment to the front portion 106 of the garment, over the shoulder areas. The FIG. 1 embodiment 100 has two quick-release upper straps 130, a first quick-release upper strap 130 and a second quick-release upper strap 131. The first and second quick-release upper straps 130, 131 may be equipped with a quick-release upper fastener loop 140 and metal or plastic hook 141 such that the post-surgical compression garment 100 may be configured to correctly fit users of varying sizes. The quick-release upper fastener loop 140 comprises a durable fabric loop 140 sewn superimposed on top of a fabric backing shoulder portion of the body, for comfort, wherein the durable fabric loop 140 forms a loop through which the metal or plastic hook 141 is able to engage, or pass through, in order to quickly and detachably engage the post-surgical compression garment 100 preferably at each shoulder area. The position of the arm holes 155, preferably beginning at a position horizontally lateral of the bottom of a user's sternum bone, can be adjusted by adjusting the adjustable quick-release fastener loop 140 and hook 141.

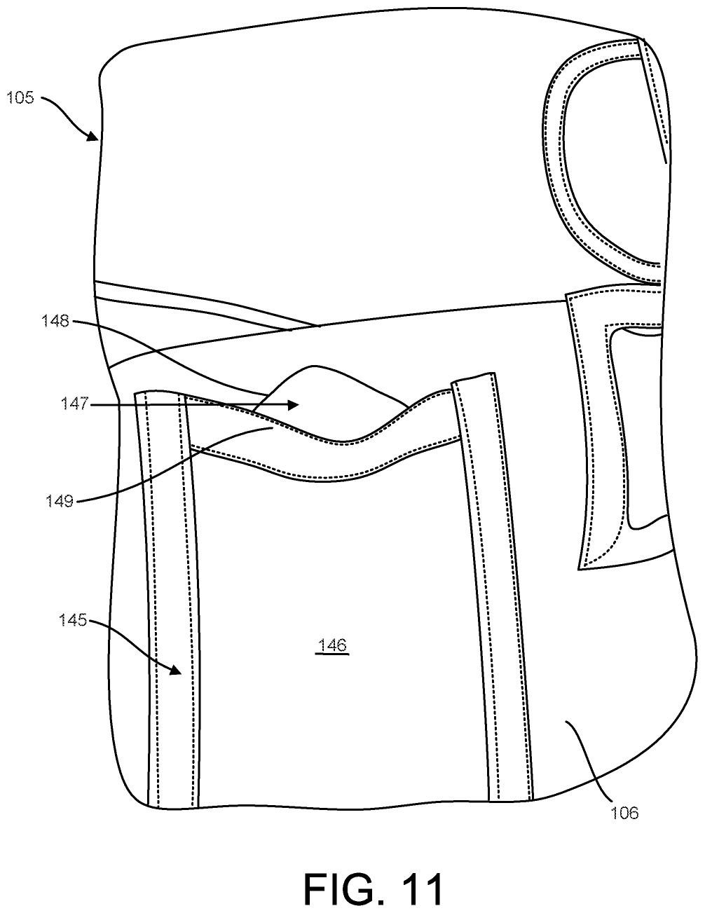

Running laterally along the garment body 105 there may be at least one quick-release tension band 120 which applies a compressive force to the user's thorax and chest, so as to keep the damaged sternum bone immobilized, in order to prevent pain, and to promote healing. Attached to the quick-release tension band 120 there may be an adjustable, quick-release tension band fastener 125 which attaches to a corresponding surface 126. The adjustable, quick-release tension band fastener 125 and its corresponding surface 126 may be comprised of hook-and-loop fasteners. The quick-release tension band 120 may optionally be equipped with a flexible band 127 connecting the quick-release tension band 120 to the quick-release tension band fastener 125 as to allow for easy closing and manipulation. As shown in FIG. 1, the quick-release tension band 120 may be positioned within a sleeve 128 which allows for the quick-release tension band 120 to move freely within the sleeve to allow adjustability of the quick-release tension band to provide an adequate compressive force to the thorax without moving the garment body 105. The embodiment in FIG. 1 is also has a telemetry pocket 145 sewn into the front portion 106 of the garment body 105 that can hold a telemetry device (not shown) or any other medical device as needed. The telemetry pocket 145 has an opening at the rear entrance thereof (as shown in FIG. 11) to allow for wires that are attached to the patient to pass through. The garment 100, as well as any of the other garment embodiments described herein, may be comprised of multiple layers of fabric to provide more compression if desired.

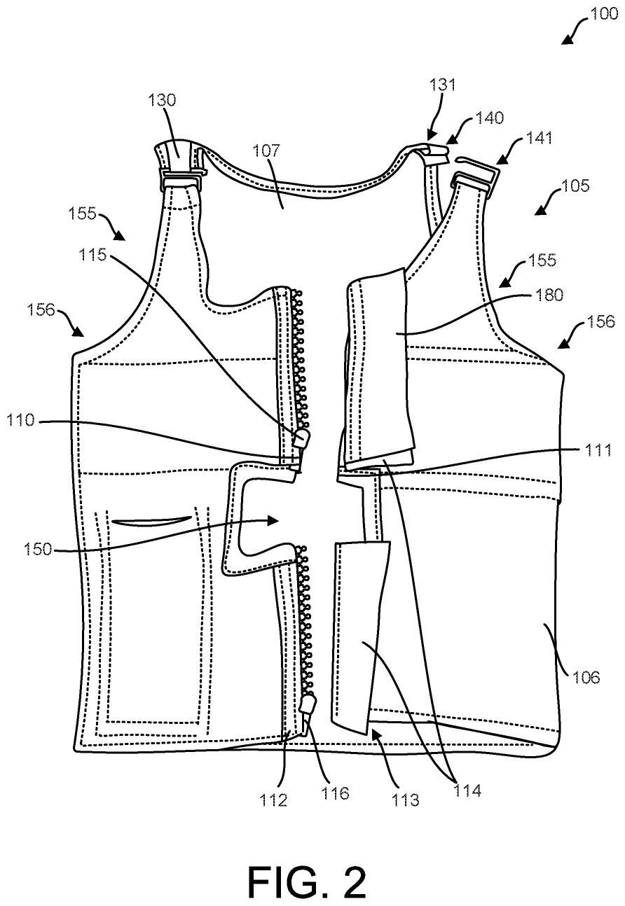

FIG. 2 shows an inside-out view of a post-surgical compression garment 100 suitable for use immediately following surgery while a patient is still in the hospital and intubated. The post-surgical thorax-compression garment 100 may be equipped with replaceable, antimicrobial pads 180 lining the interior of the front portion 106 of the garment body 105, to cover the surgical incisions, even right at a location where the garment is openable and closeable for accessibility to the surgical site, and to prevent infection. The antimicrobial pads 180 may contain a silver-based antimicrobial to prevent the buildup of bacteria and other microbes at the surface of the wound and to prevent infection. The silver-based antimicrobial may be silver metal (Ag), silver ions, silver nitrate, silver sulfadiazine, silver chloride, silver foil, silver nanoparticles, or combinations thereof. Silver nitrate in concentrations of 0.5-1% (Ag) exhibits disinfectant properties and is used for preventing infections. The antimicrobial pads 180 may also serve to promote healing and comfort by keeping the surgical incisions clean, moist, and protected from solar UV radiation and excessive airflow. The antimicrobial pads 180 may be replaceable and attached to the interior of the front portion 106 of the garment body 105 by using a pad fastener, for example a hook-and-loop fastener type, or another fastener known in the art. In some embodiments, the antimicrobial pads 180 may be washable, may retain their antimicrobial properties even upon being washed, and the same pads may later be reused without negatively impacting the wound healing process. The silver antimicrobial pads 180 may also reduce or eliminate odor from the wound, which may be attributable to the buildup of bacteria and other microbes. Various methods and techniques for incorporating antimicrobial silver compounds into wound dressings are available and described in U.S. Pat. No. 7,329,417, the disclosure of which is incorporated by reference. In one embodiment, the silver based antimicrobial pads 180 may utilize silver ions as the antimicrobial agent, as they are present in Zorb™ silver antimicrobial fabric with Silverdur™. There are further provided zipper liners 114 that wrap around the heads of the zippers 115, 116 to protect the skin from any potential friction caused by the zipper 115, 116 heads. Zipper liner 114 may be two inches in width. Other widths may be used. The front edges 110, 111, 112, 113, centrally positioned opening 150, rear portion 107 of the garment body 105, arm holes 155, bottom portion 156, quick-release upper straps 130, 131, upper fastener loop 140, and hook 141 are also illustrated.

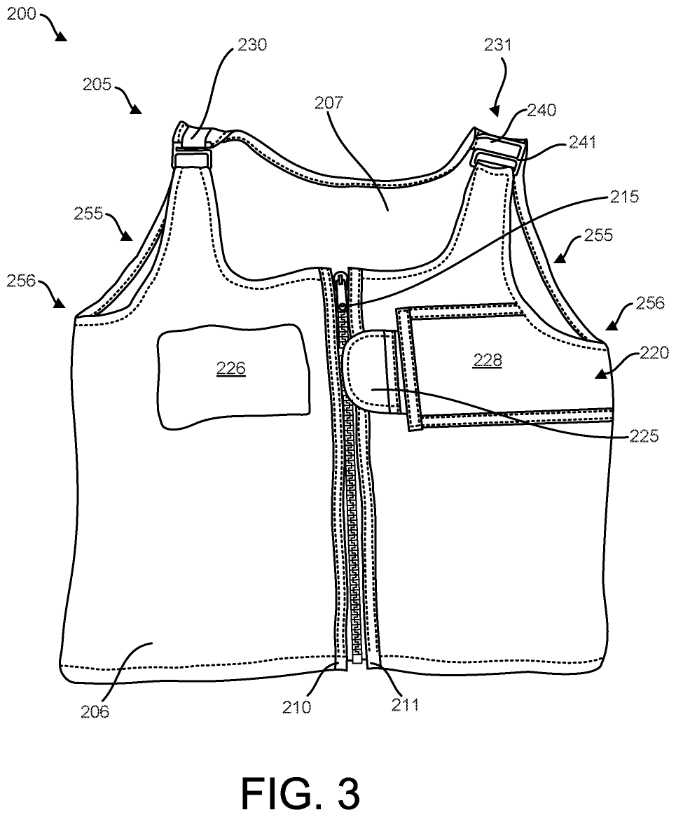

FIG. 3 shows an alternative embodiment of a post-surgical compression garment 200 suitable for home and work use in the weeks following surgery once a patient is partially recovered and no longer intubated. This embodiment of post-surgical-compression garment 200 differs from the embodiment 100 in FIGS. 1 and 2 primarily in that the garment body 205 lacks a centrally positioned opening for the passage of breathing tubes through the front portion 206 of the garment, and has only a single fastener 215 which continuously attaches the first front edge 210 and the second front edge 211 of this embodiment 200 together. The single fastener 215 may be a zipper 215.

This embodiment 200 may have a first and a second quick-release upper straps 230, 231. Each of the two quick-release upper straps may be equipped with a quick-release upper fastener 240 consisting of durable strap fabric loop 240 and metal or plastic hook 241 that is same or similar to the ones in FIGS. 1 and 2. Also shown is a single quick-release tension band 220 running laterally across the thorax, just below the bottom of the wearer's sternum bone. The quick-release tension band fastener 225 may be the same as that of the embodiment in FIG. 1. The quick-release tension band fastener 225 attaches to the corresponding band surface 226. The quick-release tension band 220 may be in a sleeve 228.

In addition to the lateral quick-release tension band 220 which provides a compressive force to the wearer's thorax, the garment body 205 itself and the single continuous fastener 215 may serve to also provide an additional compressive force to the wearer's thorax and torso, independent of the quick-release tension band 220. In such embodiments, the garment body 205 may be composed of a highly compressive fabric such as spandex and/or nylon. As a representative example, a highly compressive fabric may be composed of approximately 18% spandex and approximately 82% nylon.

As the patient recovers and becomes more able to tolerate the motion and stress at the surgical site caused by everyday movement, the patient may desire to apply less compressive force, and eventually no compressive force, using adjustability of the quick-release tension band 220, and may instead only rely upon the compressive force provided by the garment body 205. The rear portion 207 of the garment body 205, arm holes 255, and bottom portion 256 are also illustrated.

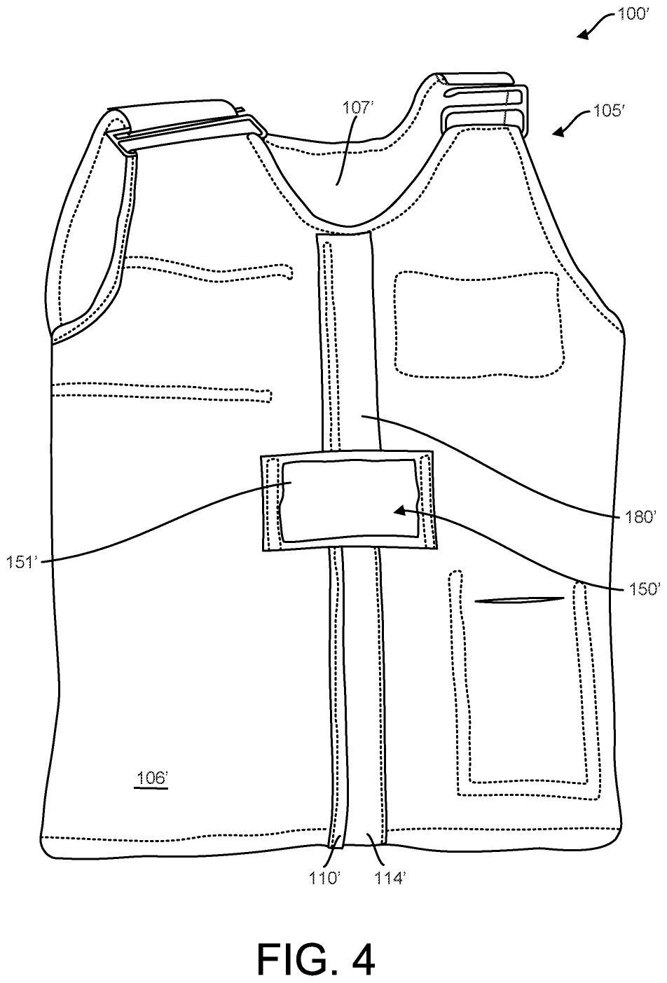

Referring to FIG. 4, an inside-out, forward view of a post-surgical compression garment 100′ suitable for home and work use in the weeks following surgery once a patient is partially recovered and no longer intubated is shown. This embodiment 100′ differs from that of FIG. 2 in that it has an opening cover 151′ which covers the centrally positioned opening 150′ in the front portion 106′ of the garment body 105′. The opening cover 151′ can attach to the centrally positioned opening 150′ by hook-and-loop fastener or any other attaching mechanism known in the art. The replaceable silver antimicrobial pad 180′ of this embodiment may also differ from the embodiment of FIG. 2 in that it may be longer, and covers a larger area, and may overlap with the opening cover 151′, covering the area over the drainage tube incision. The opening cover 151′ may also have antimicrobial pads 180′ attached. A zipper liner 114′ may be present to prevent wrap around zippers from rubbing against the skin of the wearer. Zipper liner 114′ may be two inches in width. Other widths may be used. A front edge 110′ and the rear portion 107′ of the garment are also illustrated.

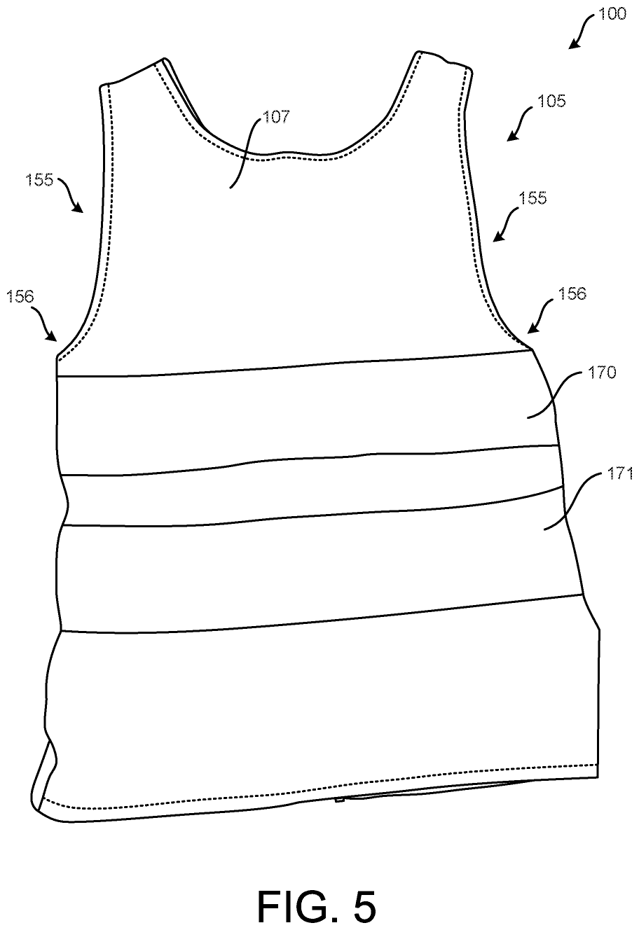

FIG. 5 shows a rear view of an embodiment of a post-surgical compression garment 100. However, this rearview is representative and can be applied to any of the embodiments. This embodiment utilizes a first and second back tension bands 170, 171 to apply a compressive force to the wearer's back and mid-back regions. The garment body 105 may be fashioned in the shape of a vest with a front portion 106 and a rear portion 107 comprising the garment body 105, which may be utilized in the any of the embodiments. In accordance with the teachings of the present disclosure, it may be possible to modify the placement of the back tension bands 170, 171 on the garment body 105 to customize any of the embodiments for a particular user. Various techniques such as stitching, gluing, or attachment using hook-and-loop fasteners to attach back tension bands 170, 171 to the garment body 105 may be used. The present embodiment illustrates the back tension bands 170, 171 as stitched into the rear portion 107 of the garment body 105. By allowing a user, or medical professional, to alter the placement of the back tension bands 170, 171 on the garment body 105, any embodiments of the post-surgical-compression garment may be customized to precisely fit the body of an individual user, and to apply compressive force exactly where a particular user desires it. In some embodiments, a large area of hook-and-loop fasteners may be placed on the rear portion 107 of the garment body 105 as to allow for the back tension bands 170, 171 to easily be moved up and down on the rear portion 107 of the garment body 105. The arm holes 155 and bottom portion 156 are also illustrated.

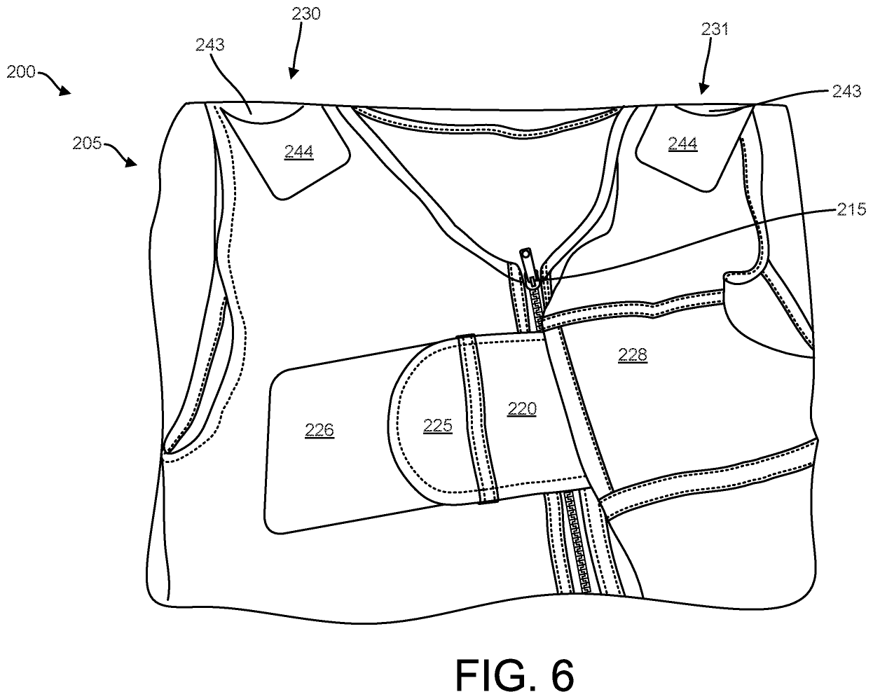

FIG. 6 shows the embodiment of a post-surgical compression garment 200 suitable for home and work use in the weeks following surgery once a patient is partially recovered and no longer intubated, wherein the quick-release tension band 220 is engaged. The quick-release tension band 220 may be enclosed within a sleeve 228 which allows for the band 220 to move freely within the sleeve 228, as to be adjusted and to provide an adequate compressive force to the thorax without moving the garment body 205. Optionally, the sleeve 228 may contain markings which indicate the level of compressive force applied by showing to what degree the quick-release tension band 220 is engaged. The quick-release mechanism for the band 220 may be a hook-and-loop fastener 225 which attaches to a corresponding band surface 226 placed upon the garment body 205. The hook-and-loop fastener 225 may be a high-strength hook-and-loop fastener.

In this embodiment, alternate versions of the quick-release upper straps 230, 231 are shown. The alternate quick-release upper straps 230, 231 may utilize high-strength hook-and-loop fasteners 243 which attach to a corresponding upper fastener surfaces 244 on the garment body 205. In such an embodiment, the alternate quick-release upper straps 230, 231 may retain their adjustability by using a corresponding upper fastener surface 243 of extended length on the garment body 205, which allows the wearer to pull the upper straps tighter by positioning them at a lower point on the front portion 206 of the garment body 205. Such an embodiment 200 may also be more stable, and desirable for use immediately following surgery while a patient is still in the hospital due to the ease of removing the high-strength hook-and-loop fasteners 243. A fastener 215, as a zipper 215, is also illustrated.

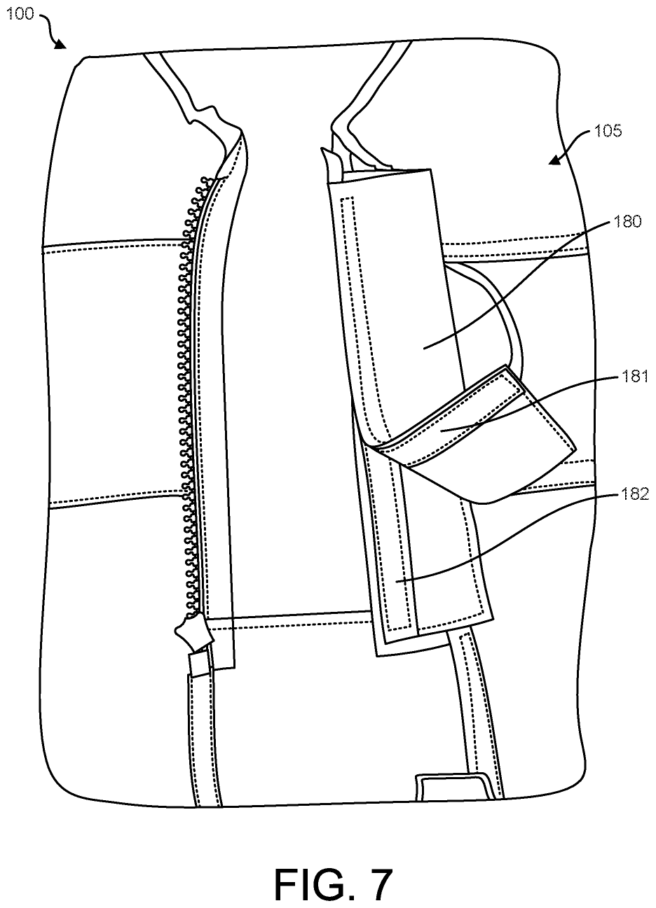

FIG. 7 shows an inside-out close-up view of the post-surgical compression garment 100 suitable for use immediately following surgery while a patient is still in the hospital and intubated, wherein the replaceable antimicrobial pad 180 is partially flipped upwards showing that it is removable. The back of the replaceable antimicrobial pad 180 will have a pad fastener 181 which permits attachment to a corresponding pad fastener surface 182 located on the interior of the garment body 105. The pad fastener 181 and its corresponding surface 182 may be hook-and-loop fasteners. Alternative methods of attaching the replaceable antimicrobial pad 180 to the garment body 105 that is known in the art, such as snaps or buttons, may also be suitable.

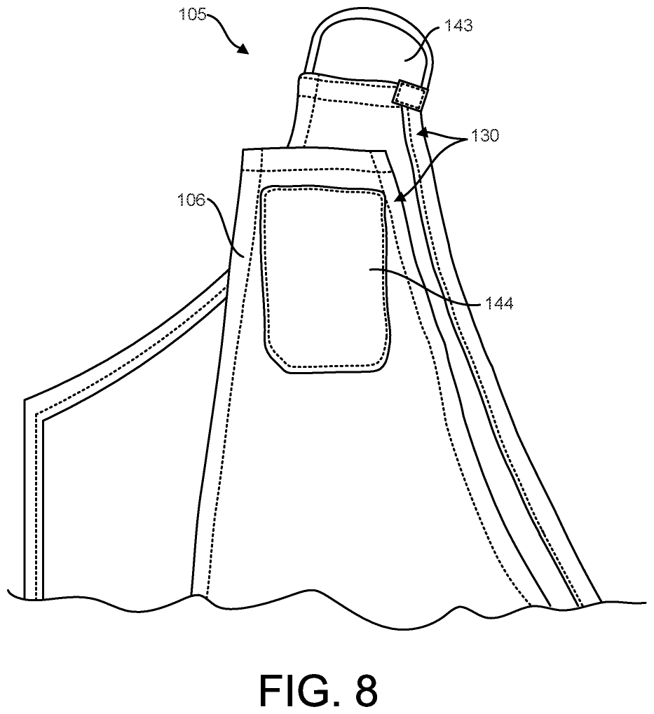

FIG. 8 shows a close-up view of the alternate quick-release upper strap 130 that may be used with any of the embodiments, which may utilize high-strength hook-and-loop fastener 143. The high-strength hook-and-loop fastener 143 may attach to a corresponding upper fastener surface 144 on the front portion 106 of the garment body 105, and may retain their adjustability by using a corresponding upper fastener surface 144 of extended length on the garment body 105, which in turn allows the wearer to adjust the tension by the moving final position of the fastener 143. A longer corresponding upper fastener surface 144 on the front portion 106 of the garment body 105 than what is pictured may be utilized in order to provide for greater adjustability.

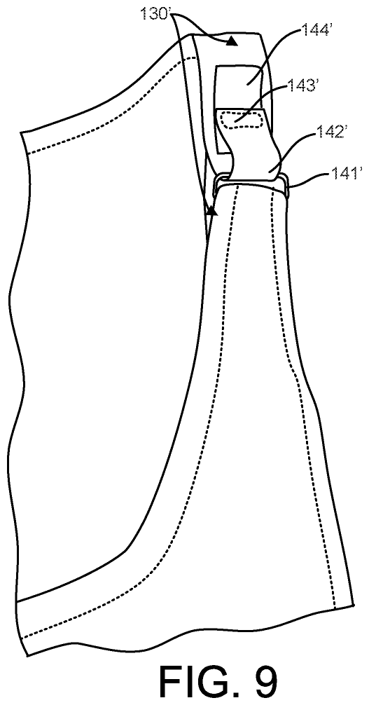

FIG. 9 shows a close-up of view of another embodiment of the quick-release upper strap 130′. In this embodiment, a metal or plastic hook 141′ is provided through which a self-adhesive long strap piece strap 142′ goes through prior to doubling back on itself to attach to itself using, for example, a hook-and-loop enabled quick-release fastener 143′ and its corresponding upper fastener surface 144′. Such an embodiment also provides adjustability for the quick-release upper strap 130 shown in FIG. 8. This quick-release upper strap 130′ embodiment may be applied to any of the garment embodiments.

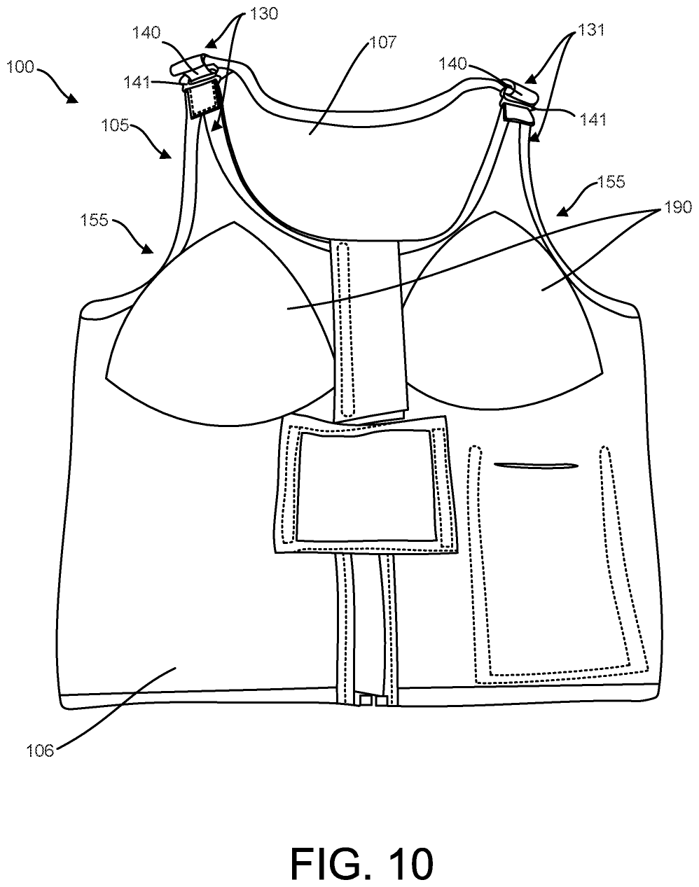

FIG. 10 shows an inside-out, forward view of a present embodiment of a garment 100, further comprising bra pads 190 on the inside portion of the front portion 106 of the garment body 105 to adapt the garment 100 for a female anatomy. Bra pads 190 may be added to any of the embodiments as desired. The size of the garment body 105, positioning of the arm holes 155, and position of the lateral quick-release tension band (not shown) may also be adjusted to apply the compressive force comfortably and appropriately, accounting for the position of breasts. Referring back to FIG. 1, the upper straps 130, 131 equipped with quick-release upper fastener loop 140 and adjustable hook 141 may be similar to hook and eye fasteners used in bras and may be ideal for a woman's garment body 105 as to allow for greater adjustability of any of the embodiments of the garment to accommodate breasts. The rear portion 107 of the garment is also illustrated.

FIG. 11 shows a close-up view of a telemetry pocket 145 of present embodiments. The telemetry pocket 145 is defined by a sewn-on outer pouch 146 and further comprises an unfastened, overlapping opening 147 in the garment body 105 which permits wires connecting to the patient to pass through the front portion 106 of the garment body 105, and to electronic monitoring devices attached to the user's chest and leading from a telemetry device (not shown) storable in the telemetry pocket 145. The overlapping opening 147 is defined by a first interior edge 148 on the inside of the front portion 106 of the garment body 105, and a second exterior edge 149 on the outside of the garment body 105, which overlaps the position of the first interior edge 148.

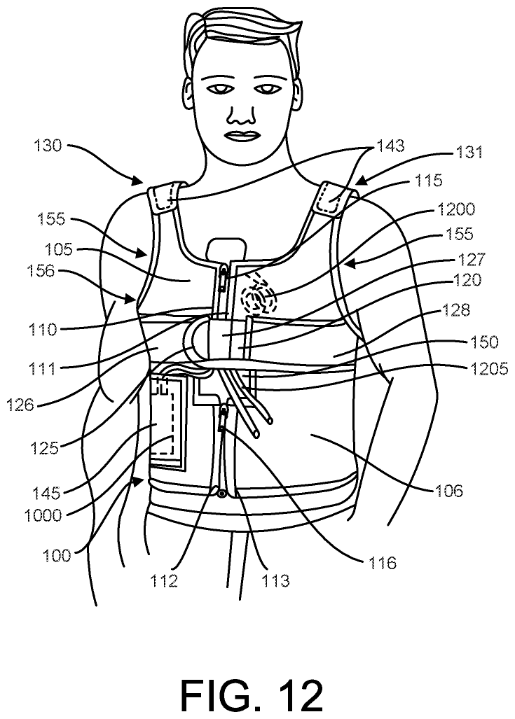

FIG. 12 shows an embodiment of a post-surgical compression garment 100 in accordance with one or more aspects of the disclosure and suitable for use immediately following surgery while a patient is still in the hospital and intubated. The arm holes 155 of the garment 100 are shown and begin at a level that is located approximately at the level of the bottom of the wearer's sternum bone. Electrical leads 1200 which are attached directly to the heart may be coiled under the garment body 105 and are to facilitate revival of a patient following open heart surgery in the event of emergency heart failure. The electrical leads 1200 are pictured in dashed lines, positioned behind the garment body 105. Also shown are drainage tubes 1205 which are inserted into the wearer's abdomen or chest wall and which are adapted to pass through the centrally positioned opening 150 in the front portion 106 of the garment body 105. Telemetry device 1000 is illustrated with dashed lines inside the telemetry pocket 145. The quick-release tension band 120 with flexible band 127 that slides into a sleeve 128 of the garment is attached with a quick-release band fastener 125 to a corresponding band surface 126 as previously discussed. The quick-release upper straps 130, 131, the quick-release upper fasteners 143, fasteners as zippers 115, 116, bottom portion 156 of the garment body 105, and the front edges 110, 111, 112, 113, are also illustrated.

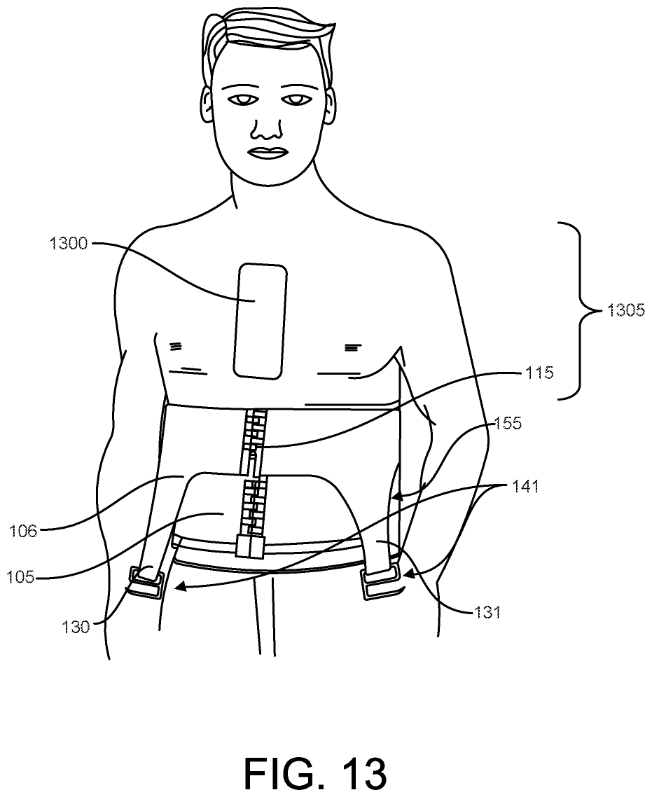

FIG. 13 shows a post-surgical compression garment of any of the embodiments in accordance with one or more aspects of the disclosure suitable for use following surgery either while a patient is still in the hospital or still recovering at home. The garment is shown having its front portion 106 of the garment body 105 flipped down. The ability of the garment to flip down and to provide quick access to the chest, surgical sites 1300, and/or electrical leads (not shown) without having to fully remove the garment, facilitates quick emergency access and thus makes it ideal for use immediately following surgery while a patient is still in the hospital and at high risk for complications or heart attack, or during recovery at home in case such quick emergency access is needed The amount of the chest and abdomen exposed 1305 should be an area great enough to provide access to the chest, surgical sites 1300, and electrical leads (not shown) in the event of an emergency. Optionally, there may be slits (not shown) in the sides of the garment body 105 at the bottom of the arm holes 155 which allow the garment to be pulled down further than otherwise possible. In this way a single sized garment may be used to achieve adequate exposure of the user's chest and abdomen for users of varying body sizes. The fastener 115, such as a zipper, the first and second quick-release upper straps 130, 131, and the hooks 141 for the upper straps are also illustrated.

The post-surgical garment of present embodiments may be fabricated from a length of high-strength compressive fabric, cut to length as required (or even doubled for greater compression), and sewn into the shape of a vest-type garment body 105. The garment body 105 is preferably cut and sewn as to account for the desired positioning of the arm holes 155 which start laterally of, and approximately at the level of, the bottom of a wearer's sternum bone. The additional attachments to the garment body 105 such as quick-release tension bands (not shown), quick-release upper strap, fasteners 115, bra pads (not shown), etc., may also be sewn to the garment body 105, but can also be glued or attached using alternative means where appropriate.

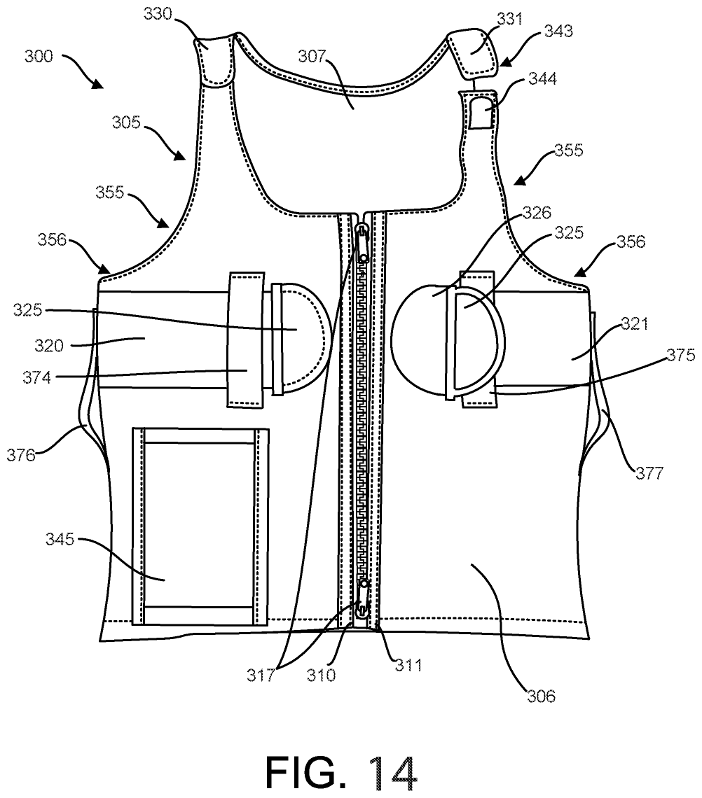

FIG. 14 shows a front view of an embodiment of a post-surgical compression garment 300 in accordance with one or more aspects of the disclosure, to be worn during recovery after a surgery to the thoracic or other torso area, such as an open-heart surgery, with a first quick-release tension band 320 and a second quick-release tension band 321 on the front portion 306 of the garment body 305. The quick-release tension bands 320, 321 may attach to the front portion 306 of the garment body 305 by quick-release tension band fasteners 325 that book onto corresponding surfaces 326 for the tension band fasteners 325. The quick-release tension band fasteners 325 and its corresponding band surface 326 (shown on one side) may be hook-and-loop fasteners. Other fasteners known in the art may be used. FIG. 14 illustrates the embodiment 300 as having a first and a second quick-release upper straps 330, 331 that utilize hook-and-loop fasteners 343 and its corresponding upper fastener surface 344 (shown on one side) to control the tension, length, and comfort of the quick-release upper straps 330, 331 as illustrated and discussed in FIG. 8.

The fastener for the garment 300 are dual-separating zippers 317. The dual-separating zippers 317 allow the wearer, caretaker, or emergency personnel to open the first and the second front edges 310, 311 of the front portion 306 of the garment body 305 without fully unzipping or fully removing the garment body 305 by either unzipping the front portion 306 of the garment body 305 from the top for easy access to the wound site or from the bottom for easy access to a lower wound site. The dual-separating zippers 317 also provide easy access in case of an emergency procedure such as resuscitation. The illustrated garment 300 also includes a telemetry pocket 345 as described in FIG. 11.

Additional new features for this present embodiment 300 include a first chest handle 374 and a second chest handle 375, placed over and adjacent to the lateral quick-release tension bands 320, 321 without hindering the movement and adjustability of the tension bands 320, 321. The chest handles 374, 375 may be placed in the midline of the chest. The chest handles 374, 375 allows a patient wearing the garment 300 to have the option of additional comfort from self-imitated compression during painful coughing, sneezing, or moving. The chest handles 374, 375 also provide caregivers and medical personnel with the ability to help move the patient when lifting or changing positions of the patient with more ease. A first side handle 376 and a second side handle 377 are also present underneath each arm holes 355. The side handles 376, 377 may be attached over the side seams of the garment body 305 and may be placed 2-inches below the arm holes 355. The side handles 376, 377 allow a caregiver or medical personnel to help stabilize the patient while the patient is moving or walking, thereby giving the caregiver or medical personnel side access and control of the patient's movement and reduce the risk of the patient falling. The chest handles 374, 375 and the side handles 376, 377 may be made of 8-inches long and 1-inch wide belt strappings and sewn or glued into the garment. The bottom portion 356 of the garment body 305 and the rear portion 307 of the garment body 305 are also illustrated.

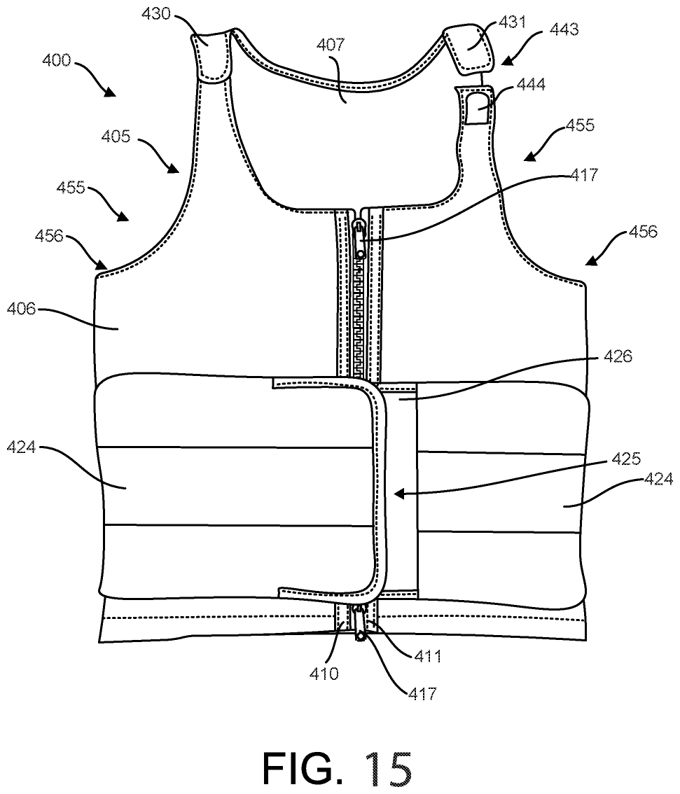

FIG. 15 shows a front view of an embodiment of a post-surgical compression garment 400 in accordance with one or more aspects of the disclosure, to be worn after an abdominal surgery. This embodiment 400 has the same features such as dual-separating zippers 417 as the embodiment 300 FIG. 14. This embodiment 400 also has the same first and second quick-release upper straps 430, 431 that utilize hook-and-loop fasteners 443 and its corresponding upper fastener surfaces 444 to control the tension, length, and comfort of the quick-release upper straps 430, 431 as illustrated and discussed in FIG. 8. Other types of quick-release fasteners known in the art may be used. Telemetry pockets (not shown), chest handles (not shown) and side handles (not shown) are removed in this embodiment 400. However, those features may be added if so desired.

To facilitate recovery after an abdominal surgery, quick tension bands as seen in previous embodiments (FIG. 1, FIG. 3, FIG. 14) are no longer desired over the thoracic area. Instead, a pair of quick-release tension band panels 424 that wrap around the abdomen of the wearer are present in the embodiment 400. The quick-release tension band panels 424 may be comprised of three 3-inches wide elastic bands placed parallel to each other and are sewn together with elastic thread. However, the size and number of elastic bands used may vary to meet the needs of the patient. For example, two 3-inch-wide elastic bands may be used for women. The elasticity of the bands also may be varied as desired. Elastic bands do not need to be 3-inches wide and can vary in size as needed for comfort and compression needed. The two quick-release tension band panels 424 overlap and attach to one another over the front portion 406 of the garment body 405. The quick-release tension band panels 424 may attach by hook-and-loop fastener 425 and its corresponding panel surface 426. Other known fasteners in the art may also be used. A fold-over bias tape may be used around the edges of the quick-release tension band panels 424. The quick-release tension band panels may be placed approximately 2-inches from the bottom of the front portion 406 of the garment body 405's hem. The first and second front edges 410, 411, the arm holes 455, the bottom portion 456 of the garment body 405, and the rear portion 407 of the garment body 405 are also illustrated.

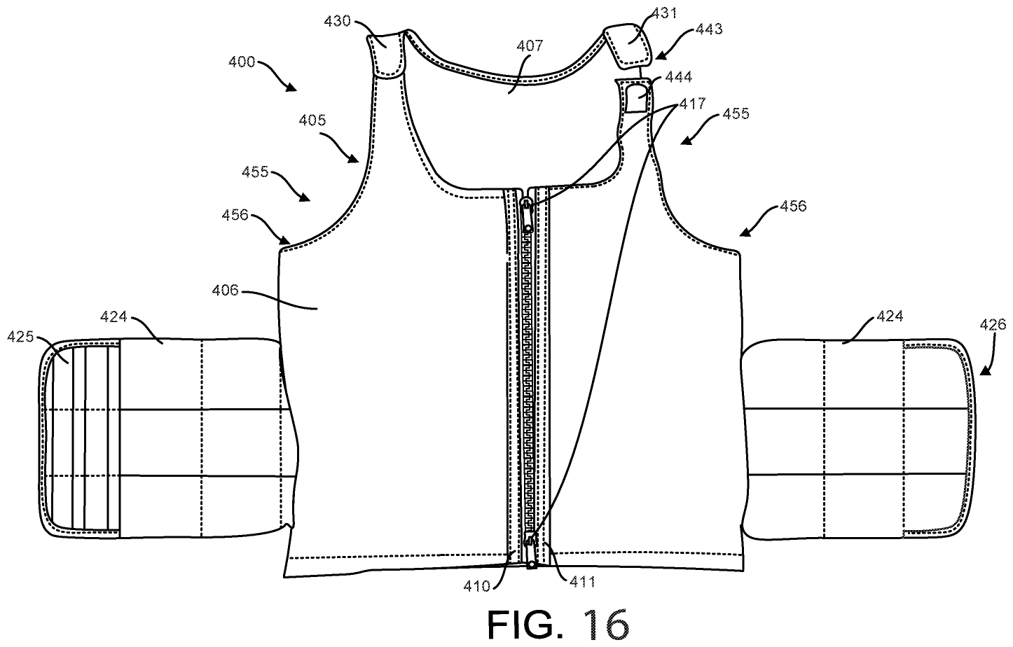

FIG. 16 shows a front view of the garment embodiment 400 of FIG. 15, with the two quick-release tension panels 424 laid open to the side of the garment body 405 to demonstrate the underside of the quick-release tension band panels 424. The quick-release tension band fastener 425 is illustrated on one side, which may be hook-and-loop fastener as discussed. The corresponding band surface 426 for the fastener is pointed to but not illustrated on FIG. 16. Other elements previously discussed in FIG. 15 are also illustrated.

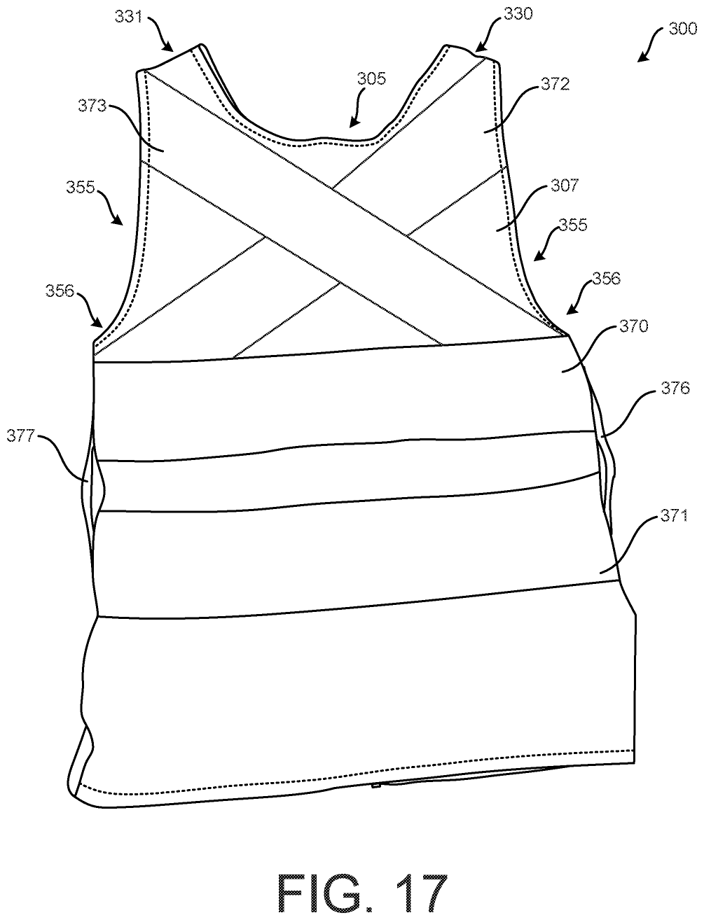

FIG. 17 shows a rear portion 307 of the garment body 305 of an embodiment 300 of a post-surgical compression garment in accordance with one or more aspects of the disclosure. This rear embodiment can be applied to any of the garment embodiments and not just the garment 300 embodiment. This embodiment of the garment 300 demonstrates a pair of back tension bands 370, 371 that compress the back and mid-back regions upon fastening or a front opening of the garment as also shown and described in FIG. 8. The back tension bands 370, 371 may be placed 2-inches apart across the back and mid-back of the wearer to provide support and pain relief from a back pain that is either inherent or possibly caused from the patient's time spent on the operating table during surgery or hospital bed during recovery. A first shoulder 5 tension band 372 and a second shoulder tension band 373 are further illustrated. The shoulder tension bands 372, 373 may cross and form an X-shape across the shoulder blades of the wearer to provide additional support and equalized tension across the upper back. This helps the patient maintain a better posture and relieve the upper back pain of the wearer. If worn after a thoracic surgery such as a heart surgery, the shoulder tension bands 372, 373 will assist in maintaining correct positioning of the sternum during its healing process. The shoulder tension bands 372, 373 may be four-inches-wide elastic bands. The shoulder tension bands 372, 373 can be of varying size and elasticity, as discussed for the back-tension bands for FIG. 8. A pair of side handles 376, 377 as described in FIG. 14 may be present but are optional to meet patient's needs. The quick-release upper straps 330, 331, arm holes 355, and bottom portion 356 of the garment body 305 are also illustrated.

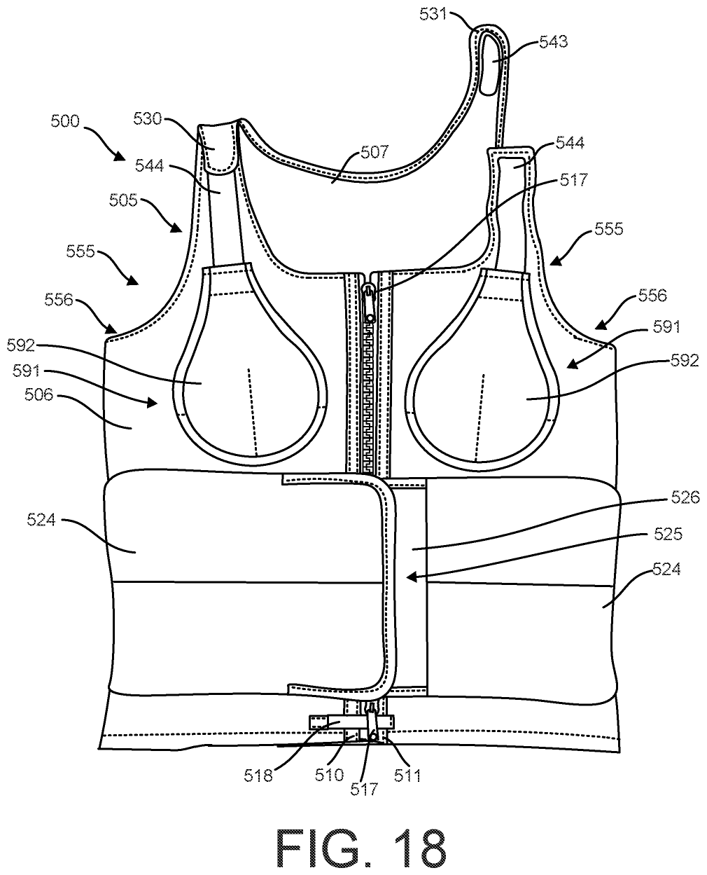

FIG. 18 shows a front view of an embodiment of a post-surgical compression garment 500 in accordance with one or more aspects of the disclosure, to be worn by a nursing mother after a Cesarean section surgery. The two quick-release tension band panels 524, like the ones described for embodiments in FIGS. 15-16, wrap around the abdomen and are held together by quick release tension band panel fastener 525 that may be a book-and-loop fastener attached to its corresponding band surface 526 as described in FIG. 15-16. The quick-release tension band panels 524 may consist of two parallel tension bands sewn together with elastic thread and as illustrated. As described in FIGS. 15-16, the size of the elastic band and the number of elastic bands used may vary to fit the wearer of varying body size. For example, three elastic bands may be used to form the quick-release tension band panels 524 to fit someone with a larger body size. Elasticity of the band may also vary as needed to accommodate different needs of patients.

The garment body 505 may be two inches longer than the abdominal compression garment shown and described in FIGS. 15 and 16. The quick-release tension band panels 524 may also be longer to accommodate this difference or shorter to accommodate breasts of varying sizes. A strip 518 of fastener may be added on the bottom hem line in the front center of the front portion 506 of the garment body 505 to keep the flap of a fastener 515, which may be a zipper or dual-separating zippers 517 as illustrated, closed. The strip 518 may also keep the two front edges 510, 511 closed when the fastener is disengaged. The fastener may be hook-and-loop fasteners or any other similar fastener type used in the art.

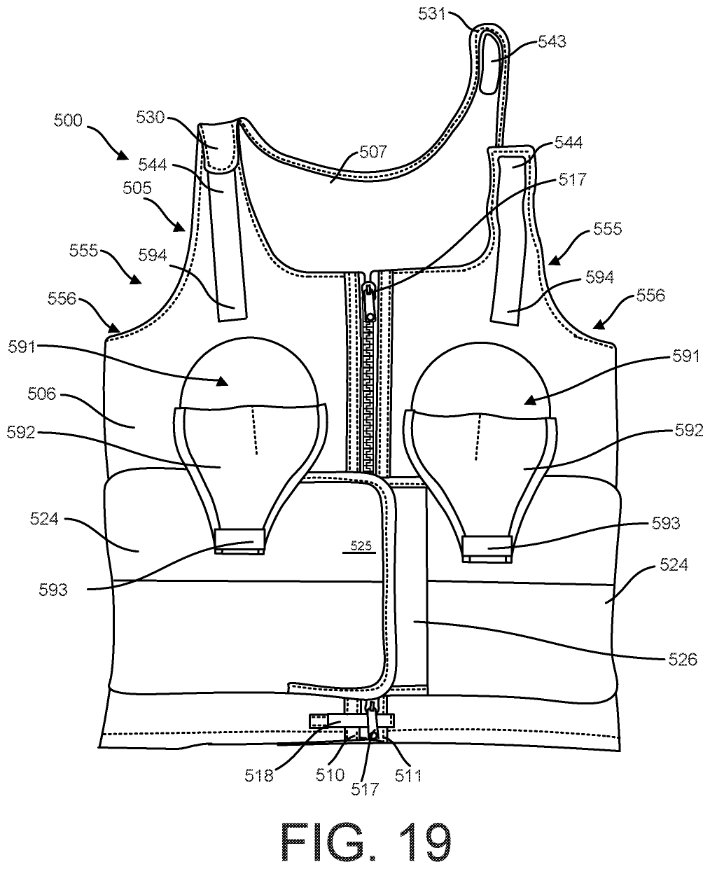

To accommodate for breast feeding, the present garment 500 has two breast cutouts 591 in the front portion 506 of the garment body 505 that are coverable by two breast covers 592. In FIG. 19, the two breast covers 592 are partially sewn, around the breast cutouts 591, onto the front portion 506 of the garment body 505 by threads. Other means, such as fasteners and glue, may be used to keep the breast covers 592 partially permanently attached to the garment 500. The breast covers 592 can also be completely detachable if so desired. As illustrated, the two breast covers 592 may partially attach and detach from the front portion 506 by breast cover fastener means, such as hook-and-loop fasteners that attach to corresponding surfaces. The quick-release upper straps 530, 531 may be comprised of any of the type of quick-release tension mechanism previously illustrated and discussed. This embodiment 500 specifically illustrates quick-release tension upper strap fasteners 543 as hook-and-loop type that attach and detach from the corresponding upper fastener surfaces 544. Further, the corresponding upper fastener surfaces 544 for the quick-release tension upper strap fasteners 543 are extended for comfort and adjustment of quick-release upper straps 530, 531 and also to allow for the breast covers 592 to also attach and detach from the same strips of corresponding surfaces 4. The breast covers 592 may attach to its own corresponding breast cover fastener surface (not illustrated) that exists separately from the quick-release upper strap fastener surfaces 544. The arm holes 555, the rear portion 507 of the garment body 505, and the bottom portion 556 of the garment body 505 are also illustrated.

FIG. 19 shows a front view of FIG. 18 embodiment 500 with the breast covers 592 flipped downwards to reveal the breast cutouts 591. The corresponding surface 594 for the breast covers 592 are the same strip of fastener as that of quick-release upper straps 544 for this embodiment but do not need to be. FIG. 19 also illustrates the breast cover fastener 593 for the breast covers 592, which is sewn or glued onto the underside of the breast covers 592. All other marked elements are the same as those illustrated and discussed in FIG. 18.

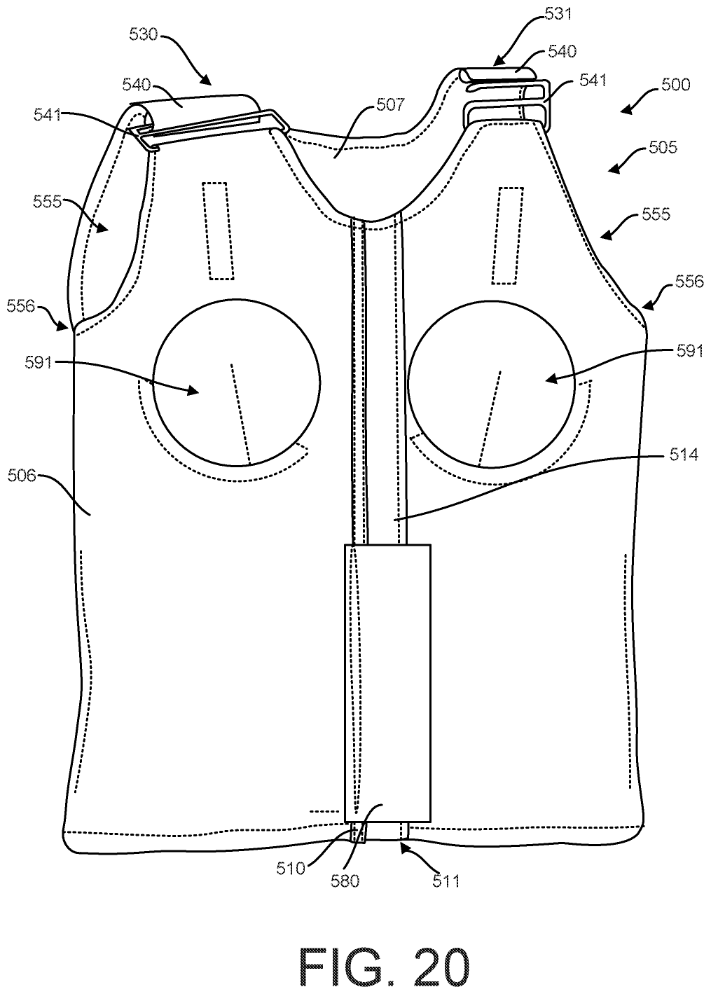

FIG. 20 shows an inside-out front view of an embodiment of a post-surgical compression garment 500 in accordance with one or more aspects of the disclosure, to be worn by a nursing mother after a Cesarean section surgery. FIG. 20 illustrates quick-release upper fastener loops 540 for the upper quick-release straps 530, 531 that hook onto a metal or plastic hook 541, as discussed and illustrated in FIGS. 1-4, to keep the quick-release upper straps 530, 531 adjusted and held in place, to 5 demonstrate that different attachment means can be used. Breast cutouts 591 are illustrated. Antimicrobial pads 580 as described previously in FIG. 2 may be attached to the abdominal area, including the area where the two front edges 510, 511 of the front portion 506 of the garment body 505 attach. Zipper liner 514, as previously discussed in FIG. 2, may wrap around the head of the fastener 515, which can be a zipper or dual-separating zippers, to protect the skin from any potential friction caused by the zipper head. Zipper liner 514 may be two inches in width. Other widths may be used without departing from the scope of the invention as claimed. The bottom portion 56 and the rear portion 507 of the garment body 505 and arm holes 555 are also illustrated.

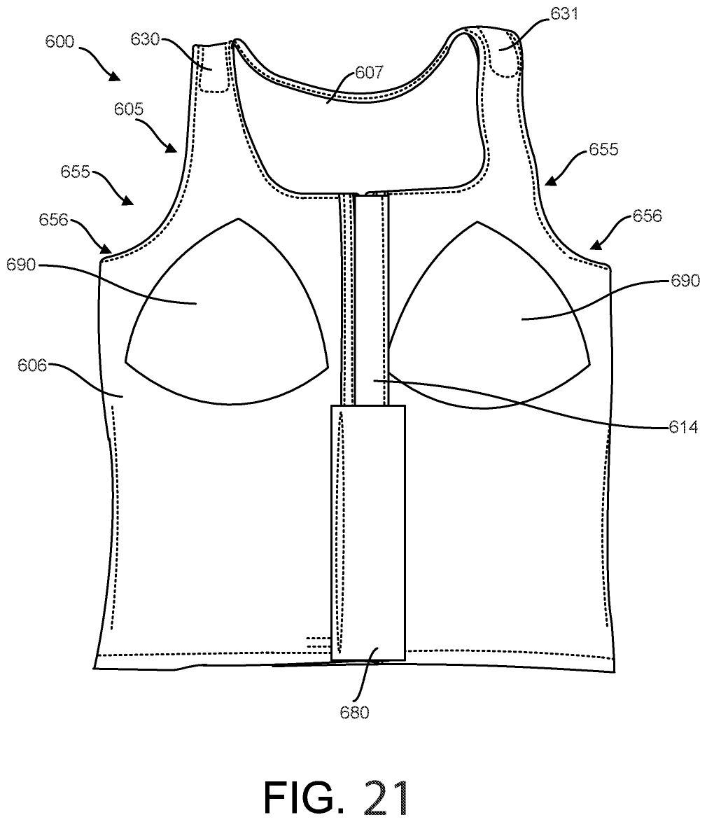

FIG. 21 shows an inside-out front view of an embodiment of a post-surgical compression garment 600 in accordance with one or more aspects of the disclosure, to be worn by a mother after a Cesarean section surgery. Instead of breast cutouts shown in FIGS. 18-20, bra pads 690, as discussed in FIG. 10, are present for the embodiment 600. The bra pads 690 can be attached to the inside of the front portion 606 of the garment body 605. Other elements of the garment 600 are the same as that of FIGS. 18-20. The quick-release upper straps 630, 631, the rear-portion 607 and the bottom portion 656 of the garment body 605, arm holes 655, antimicrobial pad 680, and the zipper liner 614 are also illustrated.

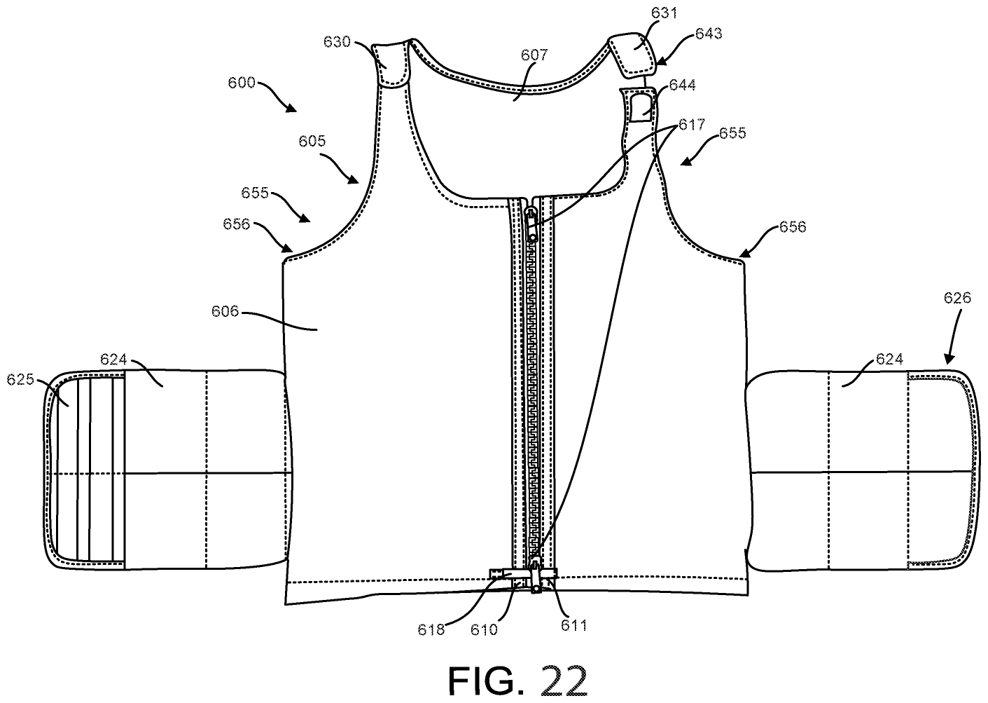

FIG. 22 shows a front view of the embodiment of a post-surgical compression garment 600 in FIG. 21 with the quick-release tension band panels 624 open to the side of the garment body 605 to demonstrate the inner side of the quick-release tension band panels 624. The quick-release tension band panels 624 attach by quick-release tension band fastener 625 that attach to the corresponding band surface 626. Other aspects are the same as that of the garment 500 of FIGS. 18-19, except that breast cutouts and breast covers are not present in the embodiment 600, and the upper strap fasteners 643 attach and detach from shorter corresponding upper fastener surfaces 644 that are used to hold the quick-release upper straps 630, 631 together. The front portion 606, the rear portion 607, and the bottom portion 656 of the garment body 605, arm holes 655, front two edges 610, 611, dual-separating zipper 617, and the strip 618 are also illustrated.

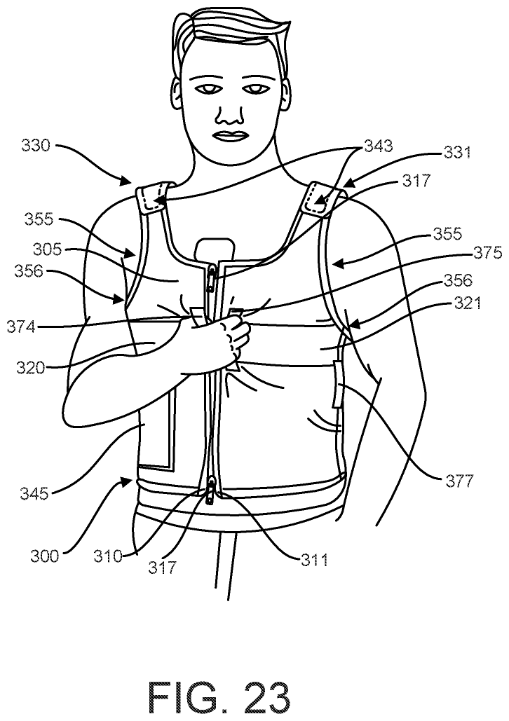

FIG. 23 shows a frontal view of a patient wearing the post-surgical compression garment embodiment 300 of FIG. 14 and engaging in self-imitated compression using the two chest handles 374, 375 by pulling the two chest handles 374, 375 together with one hand to add compression to the thorax. Self-imitated 5 compression may provide additional security and pain relief as the patient coughs, sneezes, or moves. All the visible elements from FIG. 14 are also illustrated and marked.

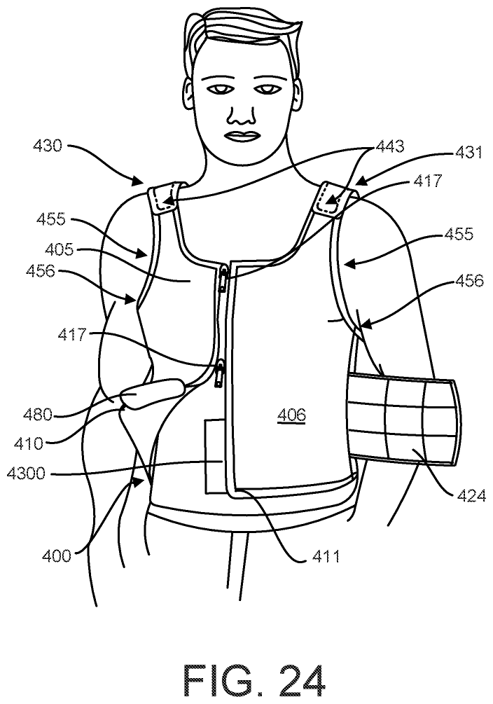

FIG. 24 shows a frontal view of a patient wearing the post-surgical compression garment embodiment 400 of FIGS. 15 and 16. The quick-release tension band panels 424 can be quickly moved aside from the front portion 406 of the garment to body 405 to reveal the first and second front edges 410, 411 that are held together by the dual-separating zippers 417. The bottom zipper of the dual-separating zippers 417 can be pulled upwards to unzip and quickly reveal the surgical site 4300 on the abdomen of the patient without removing of the whole garment 400. The surgical site 4300 can be treated or checked quickly, and the antimicrobial pad 480 can also be easily changed or removed this way. Though not illustrated, the top zipper of the dual-separating zippers 417 can be quickly pulled down from the top to flip down the front portion 406 of the garment body 405 for emergency access to the patient's chest or any surgical site that may be present in the chest area similarly as illustrated in FIG. 12. All the visible elements from FIGS. 15 and 16 are also illustrated and marked.

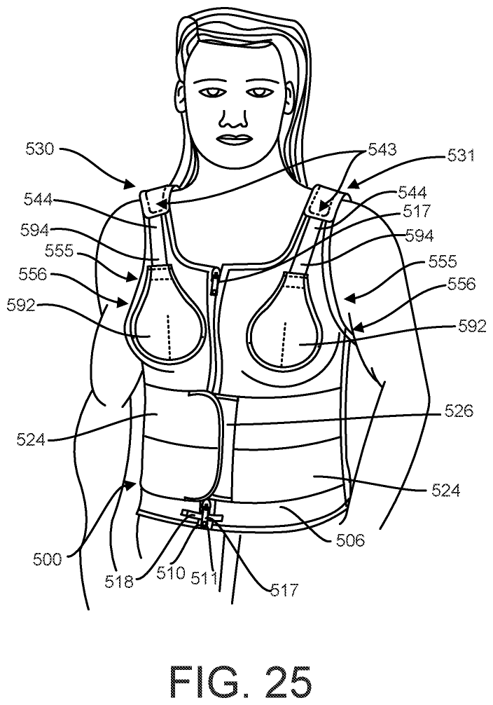

FIG. 25 shows a frontal view of a female patient wearing the post-surgical compression garment embodiment 500 of FIGS. 18 and 19 to illustrate the placement of each elements of the garment 500 on a female anatomy. All the visible elements from FIGS. 18-19 are illustrated and marked.

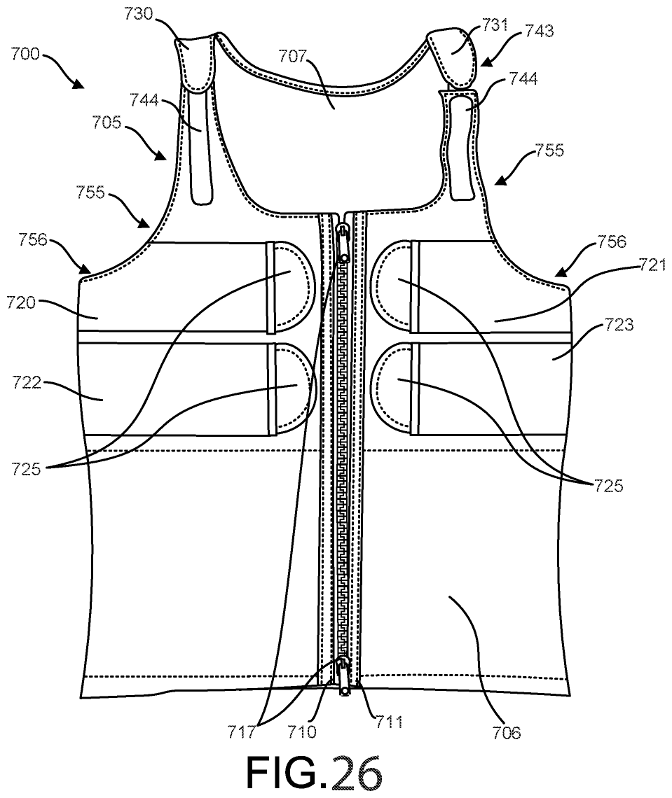

FIG. 26 shows a front view of the post-surgical compression garment 700 in accordance with one or more aspects of the disclosure, to be worn after a mastectomy. In this embodiment 700, four quick-release tension bands 720, 721, 722, 723 are laterally positioned over the breasts. The first and the second quick-release tension bands 720, 721 wrap around the upper portion of the breasts with each band 720, 721 over one breast, and the third and fourth quick-release tension bands 722, 723 wrap around the lower portion of the breasts, each band 722, 723 over one breast. The quick-release tension bands 720, 721, 722, 723 are attached to the front portion 706 of the garment body 705 by quick-release fasteners, such as hook-and-loop fasteners 725 that attach to their corresponding band surfaces, as discussed previously. Other means of attachment that is known in the art, such as buttons, can also be used. The first and the second front edges 710, 711 are held together by a fastener, which can be dual-separating zippers 717 as illustrated. Other types of fasteners known in the art may be used. The quick-release upper straps 730, 731 in FIG. 26 attach by means of hook-and-loop fasteners 743 and their corresponding upper strap fastener surfaces 744. The upper 5 strap fastener surfaces 744 are longer to allow the wearer to freely adjust the compression, length, and comfort of the garment 700, as discussed for other embodiments. As previously discussed, other types of quick-release fasteners may be used. Twill tapes may be sewn inside for additional support around the garment body 705 as needed and known in the art. Twill tapes may also be utilized in any of the embodiments. The arm holes 755, the rear portion 507 and the bottom portion 556 portion of the garment body 705 are also illustrated.

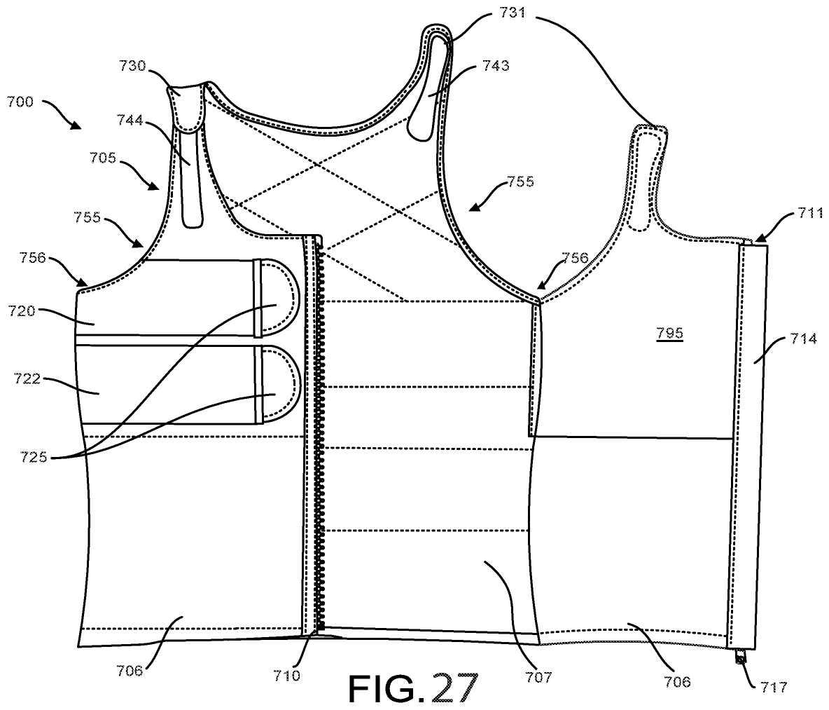

FIG. 27 shows a front view of the embodiment of the garment 700 of FIG. 26, with one front edge 711 of the garment body 705 open completely to reveal the inside of the front portion 706 of the garment body 705. The other front edge 710 is shown remaining in place to show the outside of the front portion 706 of the garment body 705. The quick-release tension bands 720, 722 that attach and detach by the quick-release tension band fasteners 725 to corresponding band surfaces are shown. The quick-release upper straps 730, 731, attach by hook-and-loop fastener 743 and an elongated corresponding upper fastener surface 744 in this embodiment 700. The zipper liner 714 that wraps around the head or heads of the dual-separating zippers 717 to protect the wearer's skin from potential friction from the zipper heads is illustrated in the side of the opened front portion 706 of the garment body 705. The zipper liner 714 may be two inches in width. Other widths may be used without departing from the scope of the invention as claimed. Additionally present is a power mesh fabric 795 over the upper region of the inside of the front portion 706 of the garment body 705. The power mesh 795 is soft and may give added breathability and comfort for extra sensitive skin caused from radiation treatments. Other similar materials known in the art may be used instead of power mesh 795. The arm holes 755, the bottom portion 756 and the rear portion 707 of the garment body 705 are also illustrated.

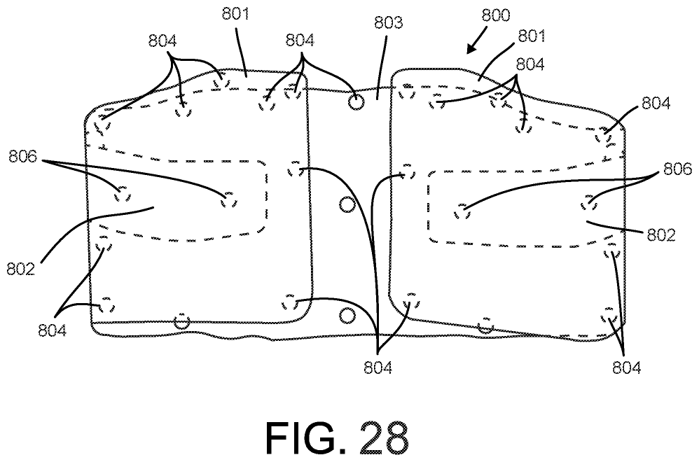

FIG. 28 shows a front perspective view of an inner lining 800, comprised of front panels 801 and a rear panel 803, for a post-surgery support garment, for example post-surgery compression garment 700, attached to the interior of the post-surgery compression garment with a hook-and-loop attachment system (or other means of attachment such as tacked with thread or clipped) 804, the lining having a pattern of antimicrobial areas 802 thereon, e.g., either infused in the lining itself or with fabric or pads detachably attached thereon with hook-and-loop attachment system 806, and adapted to be located adjacent corresponding typical incision locations for a double mastectomy. Of course, each surgery type may involve different incision patterns, and 5 therefore the lining 800 may be of different shapes and configurations to correspond with different locations within a given post-surgery support garment 100, 200, 300, 400, etc., and capable of attachment to inner portions of the particular post-surgery support garment chosen, as for example by sewing, tacking, detachable hook-and-loop attachment system 804, or otherwise (such as may be accomplished with a silk or polyester liner).

The various aforementioned embodiments and aspects of the post-surgical support garments of the invention further address the problems with the prior art since they better promote healing with a comfortable garment having a low vertical profile, which provides sufficient and appropriate compressive force to help retain an incision in place as sutured in the wearer's thorax and torso regions, which facilitates quick exposure to the patient's chest and other surgical sites by emergency personnel in the hours and days immediately following surgery and during recovery, which particularly, regarding open-heart surgery, abdominal surgery, Cesarean section surgery and mastectomy, gives such quick and easy access, and which prevents infection of surgical incisions, is disclosed.

One of ordinary skill in the art will recognize the inventive principles disclosed are not limited to the embodiments disclosed herein, and that various aspects of the disclosed embodiments can be combined to achieve additional embodiments. In the preceding description, numerous details were set forth. It will be apparent, however, to one skilled in the art, that the present disclosure may be practiced without some of these specific details.

Thus, while a preferred embodiment of the present invention has been shown and described, it will be apparent to those skilled in the art that many changes and modifications may be made without departing from the invention in its broader aspects. For example, it will be appreciated that one of ordinary skill in the art may mix and match the various components of the various embodiments of the invention without departing from the true spirit of the invention as claimed. The appended claims are therefore intended to cover all such changes and modifications as fall within the true spirit and scope of the invention.

What is claimed is:

1. A post-surgical compression garment comprising:
a garment body comprising a front garment portion and a rear garment portion, said garment body adapted to wrap at least around the torso of a wearer during use with the front garment portion positioned at least over a surgical incision site of the wearer, said garment body defining arm holes, and said garment body configured to open at the front garment portion of the garment body resulting in a first front edge and a second front edge;
a fastener for attaching the two front edges of the front garment portion of said garment body together;
at least one quick-release tension band adapted for alternately attaching and releasing tension at the front garment portion, said at least one quick-release tension band positioned laterally of the body;
at least one quick-release upper strap adapted for alternately attaching and releasing the rear of the garment to the front of the garment; and
wherein the garment body is adapted upon releasing said at least one quick-release upper strap to allow the front garment portion to flip downwards and to provide quick or emergency access to the wearer's chest without fully removing the garment.

2. The post-surgical compression garment of claim 1, wherein said at least one quick-release upper strap comprises a first quick-release upper strap and a second quick-release upper strap, each quick-release upper strap adapted to be positioned adjacent a shoulder region of the wearer during use, and wherein the garment body is adapted to allow the front garment portion to flip downwards and to provide quick or emergency access to the wearer's chest without fully removing the garment.

3. The post-surgical compression garment of claim 1 wherein said garment body is adapted to wrap at least around the thorax of a wearer during use with the front garment portion positioned at least over a surgical incision site near the sternum of the wearer.

4. The post-surgical compression garment of claim 1 further comprising replaceable antimicrobial pads attached to and lining the interior side of the garment body so as to cover the surgical incision site.

5. The post-surgical compression garment of claim 3 wherein the bottoms of the arm holes are adapted to be positioned at or below a level of a bottom portion of the wearer's sternum bone.

6. The post-surgical compression garment of claim 1 wherein the quick-release upper straps are elastic and provide tension along a vertical axis between the back of the garment body and the front of the garment body.

7. The post-surgical compression garment of claim 2 further comprising on said rear garment portion a first shoulder tension band and a second shoulder tension band, said first shoulder tension band attached and extending from said first quick-release upper strap and attached and ending at a location on the garment body below an arm hole that is located on the opposite side of the said garment body from said first quick-release upper strap, and said second shoulder tension band attached and extending from said second quick-release upper strap and attached and ending at a location on the garment body below an arm hole that is located on the opposite side of said garment body from said second quick-release upper strap, thereby said first shoulder tension band and said second shoulder tension band form a cross over the shoulder blades of a wearer to create support and equalize tension across the wearer's upper back.

8. The post-surgical compression garment of claim 1 further comprising a first back tension band positioned laterally across the rear garment portion so to lay across the back of a wearer to provide back support and a second back tension band positioned laterally across the rear garment portion so as to lay across the mid-back of a wearer to provide mid-back support.

9. The post-surgical compression garment of claim 6 wherein the quick-release mechanism of the quick-release upper straps consists of hook-and-loop fasteners.

10. The post-surgical compression garment of claim 1 wherein the quick-release mechanism of said at least one quick-release tension band consists of at least one hook-and-loop fastener.

11. The post-surgical compression garment of claim 1 wherein the fastener for attaching the first front edge and the second front edge of the garment body together is at least one zipper.

12. The post-surgical compression garment of claim 11 wherein said at least one zipper comprises dual separating zippers.

13. The post-surgical compression garment of claim 11 further comprising at least one strip of hook-and-loop fastener that attaches on the front garment portion adjacent a zipper end to keep the flap of said zipper closed at the bottom of the garment body, and an inside zipper liner attached to one of the front edges to wrap around the head of said zipper to decrease friction between the zipper and a wearer's skin.

14. The post-surgical compression garment of claim 1 further comprising at least one pocket adapted for holding a medical device.

15. The post-surgical compression garment of claim 1 further comprising an opening in the garment configured to allow for drainage tubes to extend outward from the lungs through the garment.

16. The post-surgical compression garment of claim 15, wherein said opening is centrally-located in the front garment portion such that said fastener comprises an upper fastener closing the first front edge an the second front edge of the front garment portion positioned above said opening and adapted to be located near the surgical incision site located near the sternum, further comprising a lower fastener and a third front edge and a fourth front edge, the lower fastener closing said third front edge and said fourth front edge and positioned below said opening, wherein the lower fastener is adapted to be located near a stomach portion of the wearer during use.

17. The post-surgical compression garment of claim 1 further comprising a first quick-release tension band and a second quick-release tension band positioned laterally of the body.

18. The post-surgical compression garment of claim 17 wherein said first quick-release tension band attaches adjacent to the first front edge and a second quick-release tension band attaches adjacent to the second front edge to provide equalized tension.

19. The post-surgical compression garment of claim 17 further comprising a first chest handle and a second chest handle, said first chest handle placed vertically over said first quick-release tension band adjacent said first front edge and said second chest handle placed vertically over said second quick-release tension band adjacent said second front edge, as to allow a caregiver to help adjust and move a wearer and to allow the wearer to apply self-imitated compression by pulling the first and the second chest handles together.

20. The post-surgical compression garment of claim 1 further comprising a first side handle and a second side handle, said first side handle attached below one arm hole and said second side handle attached below the other arm hole to allow stabilization and adjustment of wearer by a caregiver.

21. The post-surgical compression garment of claim 1 wherein said at least one quick-release tension band is adapted for release to facilitate lowering of said front garment portion.

22. The post-surgical compression garment of claim 1 further comprising bra pads adapted for female anatomy.

23. The post-surgical compression garment of claim 1 further adapted for female anatomy, comprising two breast cutouts on said front garment portion that allows for exposure of breasts, two breast covers attached to said front portion to cover said breast cutouts and adapted to flip downwards to reveal said breast cutouts to allow for breast feeding without removal of the said post-surgical compression garment.

24. The post-surgical compression garment of claim 16, wherein both the upper fastener and the lower fastener each comprise a zipper.

25. The post-surgical compression garment of claim 1, wherein at least one quick-release tension band is multiple quick-release tension bands that are sewn together to form two panels that are each attached to opposite sides below respective arm holes of said garment body, said two panels wrap around the abdomen of a wearer and attach together over said first front edge and said second front edge.

26. A post-surgical compression garment of claim 25, wherein the panels attach by a hook-and-loop fastener.

27. A post-surgical compression garment adapted for wear immediately after open-heart surgery adapted to assist with alleviating pain, to accommodate drainage tubes, to accommodate retention of heart monitoring apparatus near a patient's chest, and to accommodate quick access by medical personnel to leads attached directly to the heart in case of post-surgery heart attack, comprising:
a garment body comprising a front garment portion and a rear garment portion, said garment body adapted to wrap around the thorax and abdomen of the patient during use with a front garment portion divided into upper and lower front garment portions, the upper front garment portion adapted to be worn adjacent a surgical incision site in the patient's chest, and the lower front garment portion adapted to be worn adjacent the patient's abdomen, said garment body defining arm holes having lowermost portions adapted to be positioned at a level just below the patient's sternum, said garment body configured to open at the front garment portion of the garment body resulting in four front edges;
an opening centrally positioned between the upper and lower front garment portions and adapted to allow for drainage tubes to extend outward from the lungs through the garment;
a first zippered fastener for attaching two of the front edges of the upper front garment portion;
a second zippered fastener for attaching other two of the front edges of the lower front garment portion;
a plurality of quick-release tension bands each adapted for alternately attaching and releasing tension at the front garment portion, at least one quick-release tension band adapted for use with the upper front garment portion, and at least one quick-release tension band adapted for use with the lower front garment portion, said plurality of quick-release tension bands positioned laterally of the body;
a plurality of quick-release upper straps adapted for alternately attaching and releasing the rear garment portion to the front garment portion, at least one quick-release upper strap adapted for use adjacent a left shoulder of the patient during use, and at least one quick-release upper strap adapted for use adjacent a right shoulder of the patient during use;
at least one pocket sewn into the front garment portion adapted for holding a heart monitoring device;
wherein the garment body is adapted, upon releasing said plurality of quick-release upper straps, and upon releasing said at least one quick-release tension band adapted for use with the upper front garment portion, to facilitate allowing the front garment portion to be quickly flipped downward away from the surgical incision site to provide quick emergency access to heart leads taped to the wearer's chest beneath the upper front garment portion and without necessitating full removal or full opening of the garment.

28. The post-surgical compression garment of claim 1 wherein the garment body comprises cotton, polyester, spandex, nylon, modal, or silk and is machine washable.

29. The post-surgical compression garment of claim 16 further comprising bra pads adapted for female anatomy.

30. The post-surgical compression garment of claim 16, wherein said quick-release upper straps are elastic and provide tension along a vertical axis between the back garment portion and the front garment portion of the garment body, and wherein said quick-release tension bands are elastic and provide tension along a horizontal axis across the upper front garment portion and the lower front garment portion, and wherein releasing of said quick-release upper straps and releasing of said quick-release tension band near the upper front garment portion is adapted to enable quick emergency access to the patient's chest by facilitating lowering of the upper front garment portion and without necessitating unzipping of either the first or the second fasteners.

31. A post-surgical compression garment comprising:
a garment body comprising a front garment portion and a rear garment portion, said garment body adapted to wrap at least around the abdomen of a wearer during use with the front garment portion positioned at least over a surgical incision site, said garment body defining arm holes, and said garment body configured to open at the front garment portion of the garment body resulting in two front edges;
a fastener for attaching the two front edges of the front garment portion of said garment body together; and
at least one quick-release tension band adapted for alternately attaching and releasing tension at the front garment portion, said at least one quick-release tension band positioned laterally of the body;
wherein the garment body is adapted upon releasing said at least one quick-release upper strap to allow the front garment portion to flip downwards and to provide quick or emergency access to the wearer's abdomen without fully removing the garment.

32. The post-surgical compression garment of claim 31
wherein said at least one quick-release tension band comprises a plurality of quick-release tension bands that are sewn together to form two panels that are each attached to opposite sides below respective arm holes of said garment body, said two panels wrap around the abdomen of the wearer and attach together over said front two edges of the said post-surgical compression garment wherein the panels attach by a hook-and-loop fastener.

33. The post-surgical garment of claim 1, wherein at least one quick-release tension bands comprise a first, a second, a third, and a fourth quick-release tension bands positioned laterally across the breasts.

34. The post-surgical garment of claim 33, further comprising a power mesh fabric attached to the upper portion of the inside of said front garment portion for breathability and comfort.
